(19) United States
(12) Patent Application Publication
(10) Pub. No.: US 2025/0009664 A1
(43) Pub. Date: Jan. 9, 2025

(54) DOSAGE FORM FOR INTRA-ARTICULAR INJECTION COMPRISING COLCHICINE FOR USE IN THE TREATMENT OF CRYSTAL-AND NON-CRYSTAL ASSOCIATED ACUTE INFLAMMATORY ARTHRITIS

(71) Applicant: PK MED, Paris (FR)

(72) Inventors: Charles SANSON, Paris (FR); Daniel HAYOZ, Paris (FR); Hang-Korng EA, Paris (FR); Gauthier POULIQUEN, Paris (FR)

(21) Appl. No.: 18/576,452

(22) PCT Filed: Jul. 12, 2022
(86) PCT No.: PCT/EP2022/069416

§ 371 Date: Jan. 4, 2024

(30) Foreign Application Priority Data
Jul. 19, 2021 (EP) 21306016.3

(51) Int. Cl. A61K 9/16; A61K 9/00; A61K 9/10; A61K 31/165; A61P 19/02

(52) U.S. Cl. CPC A61K 9/1647; A61K 9/0019; A61K 9/10; A61K 31/165; A61P 19/02

(57) ABSTRACT

The present invention relates to pharmaceutical composition under the form of a suspension comprising controlled release microparticles comprising colchicine for use in the treatment of crystal-associated and non-crystal associated acute inflammatory arthritis by intra-articular injection into a joint of said pharmaceutical composition, wherein the microparticles have a mean particle size greater than 10 pm, wherein the time required to release 80% by weight of the colchicine in the microparticles is greater than 1 day and may reach 15 days, wherein the colchicine is present in a concentration ranging from 2.5 to 2500 pg per ml of suspension and wherein the pharmaceutical composition has a volume ranging from 0.1 ml to 5 ml.

The present invention relates to the field of the treatment of inflammatory pain in joints by colchicine. Inflammatory pain in joints aimed at in the present invention may be crystal-associated and non-crystal associated acute arthritis, in particular arthropathies attacks or flares, and acute forms of tendinitis and capsulitis and merely targets acute diseases. Colchicine is administered locally by intra articular injection via a controlled release dosage form.

### BACKGROUND

Inflammatory arthritis are a group of chronic auto-immune and auto-inflammatory diseases characterized by joint inflammation with symptoms such as pain, swelling, stiffness and decreased range of motion. Patients generally experience alternating acute periods with highly intense symptoms in one or several joints and periods of inactivity. Inflammatory arthritis includes several diseases and conditions such as Rheumatoid arthritis, Ankylosing spondylitis, Juvenile arthritis, Psoriatic arthritis, Lupus or Microcrystalline diseases.

In particular, the class of microcrystalline arthropathies corresponds to an inflammation of local tissue caused by the deposition of microcrystals in joints. These crystals trigger an inflammatory reaction and severe joints pain. This class of diseases includes several conditions which differ in particular in the nature of the crystals: Gout (monosodium urate—MSU), chondrocalcinosis (calcium pyrophosphate—CPP), calcifying tendinitis (calcium phosphate) and Milwaukee shoulder syndrome (calcium phosphate). In this context, the crystal-associated arthropathies are characterized by multiple, extremely painful acute attacks or flares followed by pain, disability and impaired quality of life.

Tendinitis is an inflammation of a tendon that can be secondary to several factors including traumatic, degenerative, iatrogenic i.e. drug-induced, age and inflammation, responsible of pain and disability. Tendinitis can occur at any age, but it is more common among adults who do a lot of sport or any intense cyclic tractions on the tendon. Older people are also susceptible, because the tendons tend to lose elasticity and become weaker with age. A chronic pain presentation also exists in calcifying tendinitis, as well as an acute form of calcifying tendinitis.

Capsulitis is an inflammation of a capsule generating pain and stiffness. Capsulitis frequently affects the shoulder and, in this case, may also be called frozen shoulder.

For tendinitis and capsulitis, the present invention mainly targets their acute form and for example calcifying tendinitis.

Treatment recommendations for these acute joint diseases are most often symptomatic and aim to control the pain. Generally, the first line options are mainly oral nonsteroidal anti-inflammatory drugs (NSAIDs), oral or intra-articular corticosteroids or oral colchicine (reglementary approved for gout). When it is possible and when the joints are accessible, i.e. in the case of large joints (knees, ankles, wrists, elbows and shoulders), intra-articular corticosteroid injection is considered by practitioners to avoid systemic administration and thus avoid the risk of significant side effects.

Intra-articular administration of corticosteroid, used for several decades in joint diseases in general, permits an improvement in pain and mobility, with non-lasting effects (a few weeks in the case of an immediate release form). However, the toxicological aspect of these administrations is quite problematic and also controversial. Indeed, systemic effects are mentioned for weeks following the injection including Cushing's syndrome, hyperglycemia, decreased bone density or a risk of infections. The latter being the consequence of the direct introduction of an infectious agent into the articulation or by reducing the immune response and allowing a latent tuberculosis infection to be reactivated. This type of treatment is in particular not adapted for patients at risk, i.e., for example with comorbidities, like diabetes or osteoporosis. In addition, deleterious effects are also described on local tissue as damage to articular cartilage and tendons.

Consequently, in medical practice, the frequency and dose of intra-articular corticosteroid injections are limited. For the immediate release galenic forms it is therefore recommended a maximum of three injections per year/per site and a minimum interval of three months between two injections. A first intra-articular sustained-release formulation of corticosteroid (Zilretta® by Flexion Therapeutics) was approved in 2017 by the FDA for osteoarthritis, allowing continuous treatment for months. However, this product is not approved for repeated administrations.

Intra-articular administration of corticosteroids therefore does not seem to be an optimal treatment for extremely painful attacks, such as gout attack or chondrocalcinosis.

Colchicine is a drug characterized by pleiotropic effects related to its capacity to bind to tubulins and disturb microtubule polymerization, destabilizing the cytoskeleton and all the cellular processes, including cell division, migration and cell shape. Among these effects, colchicine affects the immune system and downregulates multiple inflammatory pathways, leading to a decrease in neutrophil function and migration. Colchicine is therefore used and approved in several inflammatory diseases such as Familial Mediterranean Fever, Behget disease and Microcrystalline diseases.

Colchicine has indeed been used for a very long time for the non-specific treatment of crystal-associated arthropathies. In the past 50 years, the use of short courses of fast-acting NSAIDs and corticoids have been considered with colchicine as the oral drugs of choice for the symptomatic treatment of acute gout and pseudogout. In the article George Nuki, MB, FRCP “Colchicine: its Mechanism of Action and Efficacy in Crystal-Induced Inflammation”, Current Rheumatology Reports, 2008, 10:218-227, prophylactic and therapeutic efficacy of oral administration of colchicine is reviewed. Still in said article, side effects of colchicine are extensively described, such as powerful purgative effects, bone marrow suppression, neuromyopathy and rhabdomyolysis, especially in patients with renal insufficiency as well as high mortality associated with overdosage.

Colchicine has thus an exceptionally narrow therapeutic index. Therefore, for all these reasons, the use of colchicine remains limited although its efficacy has been largely proven.

Moreover, although it is known to treat inflammation in joints, i.e. at site, by directly administering an active substance therein, it has been found that the active substance escapes relatively easily from said joint, thus reducing the therapeutic effect.

Colchicine encapsulation for sustained release has been reported in different carriers, such as in polymeric microspheres (Das, G. S. et al. “Colchicine Encapsulation within Poly(Ethylene Glycol)-Coated Poly(Lactic Acid)/Poly(ε-Caprolactone) Microspheres-Controlled Release Studies” Drug Delivery, 7: 7544, 129-138 (2000)). In this article, colchicine has been investigated in the treatment of other diseases, such as restenosis after angioplasty or vascular injury, and on this occasion, attempts of encapsulation of colchicine within poly(lactic acid)/poly(ε-Caprolactone) microspheres were performed for local delivery of colchicine. Another composition comprising biodegradable microparticles containing colchicine was further described seeking the treatment of the same pathology, i.e. restenosis, through intramural delivery, as reported in Gradus-Pizlo Irmina et al: “local delivery of biodegradable microcapsules containing colchicine or a colchicine analogue: effects on restenosis and implications of catheter-based drug delivery”, Journal of The American College of Cardiology, vol. 26, no. 6, pages 1549-1557. It clearly comes out that the therapeutic target is completely different from the one of the present invention. In addition, the results are not encouraging and point out toxicity risks. Indeed, signs of local toxicity were observed in the muscle layers overlying and fed by arteries infused with the microparticles containing colchicine or colchicine analogue but not control microparticles. For both disclosures, it is furthermore worth to notice that the particle size is lower than the one of the particles implemented in the present invention, as it will be apparent in the following description.

In addition, colchicine has previously been reported in U.S. Pat. No. 5,747,060, again for other effects than the one as directed to in the framework of the present invention, as acting on the prolongation of the duration of the anesthetic effect produced by the local administration of an anesthetic. Colchicine is thus co-administered in example 1 of said document around the sciatic nerve with bupivacaine, under an immediate release form, i.e. directly added into an aqueous solution.

WO2006/066419 discloses the same effect of colchicine with respect to the prolongation of its anesthetic effect and namely a combination of a vanilloid receptor agonist and another molecule, possibly colchicine. An example discloses a composition comprising a mixture of resiniferatoxin and colchicine injected intra-articularly for the treatment of capsulitis. Said injection is however additionally dedicated for an immediate release not seeking to control the local profile and systemic pharmacokinetics of colchicine, nor to extend its therapeutic exposure.

The use of colchicine for the treatment of crystal-associated arthropathies is well established. However, nowadays, colchicine is only commercialized under an immediate release oral dosage form, classically with a daily dosage for the treatment of the crystal-associated arthropathy around 1 mg per day. The FDA-approved dosage for the treatment of acute gout flare is 1.2 mg colchicine at the first sign of the flare followed by 0.6 mg one hour late (https://www.fda.gov/drugs/postmarket-drug-safetv-infornation-paticnts-and-providers/colchicine-marketed-colcrys-information). The recommended colchicine first-line treatment for acute flare by EULAR is a loading dose of 1 mg followed 1 hour later by 0.5 mg (Richette, P. et al. 2016 updated EULAR evidence-based recommendations for the management of gout. Ann. Rheum. Dis. 76, 29-42 (2017)). However, as explained above, and illustrated herein after in more details, reaching colchicine therapeutically active dose locally is hindered by the low tolerance or high toxicity of orally administered colchicine rendering the drug prescription extremely complicated.

Colchicine was recently described in a nanoemulsion system injected into the joint of Swiss albino mice model with MSU (Monosodium Urate) crystal-induced gouty arthritis: Aboumanei et al, “intra-articular formulation of colchicine loaded nanoemulsion systems for enhanced locoregional drug delivery: In vitro characterization, ^{99m}Tc coupling and in vivo biodistribution studies”, DOI: 10.1080/03639045.2021.1934865. The biodistribution pattern of ^{99m}Tc-ColNE-5 as well as ^{99m}Tc-Col solution (^{99m}TcColS) was studied. However, said disclosure is limited to an observation of a radiolabeled colchicine which is thus a different molecule from colchicine itself and within a period not exceeding 24 hours. It is well known that labelling with radionuclide like ^{99m}Tc can have a strong impact on the pattern of biodistribution of the labelled entity due to modification of its charge, lipophilicity and stability (Decristoforo et al. “The influence of chelator on the pharmacokinetics of 99mTc-labelled peptides.” Q. J. NUCL. MED. 46 3 (2002): 195-205). Moreover, said article is silent on the technical problem of narrow therapeutic index as explained above and very specific to the colchicine, i.e. the maximal systemic concentration to be avoided for toxicity reasons. Said article is thus also silent on microparticulate systems with higher sizes and targeted dosages able to reach effective local concentration for combatting the acute forms of the considered pathologies, with no toxicity at a systemic level. It namely appears in FIG. 3 of said article that ColNE-5, i.e., «colchicine nanoemulsion system» provides a release of 40% of colchicine in less than 3 hours, which is contrary to the fulfilment of a controlled systemic concentration of colchicine for avoiding side effects, as required in the present invention. Achieving release profiles not having such a quick release just after the administration, also called “burst release”, as explained herein after, that can be detrimental in terms of side effects in such an extent, implies as such a non-obvious approach which was not predicable from said article. In other words, no colchicine controlled release profile suitable for human healing is revealed in said article, and even more particularly for the treatment of a crystal-associated or non-crystal associated acute inflammatory arthritis, in particular effective in treating non-crystal associated acute arthritis, such as Rheumatoid arthritis, Ankylosing spondylitis, Juvenile arthritis, Psoriatic arthritis, Lupus or in treating crystal-associated arthropathies attacks or flares, such as Gout, Chondrocalcinosis, calcifying tendinitis and Milwaukee shoulder syndrome.

IV injection of colchicine is considered effective in the treatment of acute gout attack with a dose not exceeding 2-3 mg per single injection (Nuki, G. Colchicine: Its mechanism of action and efficacy in crystal-induced inflammation. Curr. Rheumatol. Rep. 10, 218-227 (2008)). However even if GI side effects can be avoided using IV administration, severe toxicity was associated with inappropriate dosing (Wallace S L, S. J. Review: systemic toxicity associated with the intravenous administration of colchicine-guidelines for use. J Rheumatol. 15, 495-9 (1988), Bonnel, R. A., Villalba, M. L., Karwoski, C. B. & Beitz, J. Deaths associated with inappropriate intravenous colchicine administration. J. Emerg. Med. 22, 385-387 (2002)). This led authorities to eliminate the use of IV colchicine for acute gout treatment (Zhang, W. et al. EULAR evidence-based recommendations for gout. Part II: Management. Report of a task force of the EULAR Standing Committee for International Clinical Studies Including Therapeutics (ESCISIT). Ann. Rheum. Dis. 65, 1312-1324 (2006)). Attempts of i.v. injection of colchicine have furthermore largely discouraged the man skilled in the art to envision injection of colchicine, as reported in E. Niel et al, “Colchicine today”, Joint Bone Spine 73 (2006) 672-678.

Therefore, it remains a need to develop novel dosage forms, route of administrations and targeted dosage for colchicine, both for obviating the side-effects, in particular observed during systemic administration, and improving the efficacy by optimizing the available dose of colchicine, in particular for extending its local duration of action at the painful site, in particular by increasing the local concentration, in particular for the treatment of acute crisis of crystal-associated inflammatory arthritis and non-crystal associated inflammatory arthritis.

The invention has for purpose to meet the needs, thereby both obtaining increased advantage from its action against the inflammation and restricting the level of any side effects.

One of the advantages of the present invention is to provide a new mean to achieve a particular colchicine release profile which respects an adequate ratio between the local or synovial concentration of colchicine and the systemic concentration thereof, suitable for offering the best therapeutic action locally and at the same time avoiding potential systemic toxicity.

Due to the reduced side-effects obtained by the administration of the pharmaceutical composition according to the present invention, it is furthermore particularly suitable for people suffering from chronic comorbidities (Hypertension, Chronic Kidney diseases, Diabetes, Heart diseases, Immunosuppression . . . ) fairly prevalent conditions in microcrystalline joint diseases (50% of Gout patients having at least 3 comorbidities). Indeed, the management of microcrystalline joint diseases patients having multiple comorbidities, remains a challenge for medical practitioners due to frequent and major contraindications to either Colchicine, NSAIDS or Corticosteroids. In comparison to the existing oral colchicine treatment, the present invention, in addition to avoid gastrointestinal side effects, prevents major toxicity risks for patients suffering from renal or hepatic impairments or for co-medicated patients with CYP3A4/PGP drug inhibitors, which are fairly prevalent medications.

### SUMMARY OF THE INVENTION

According to a first aspect, herein is provided a pharmaceutical composition under the form of a suspension comprising controlled release microparticles comprising colchicine for use in the treatment of crystal-associated and non-crystal associated acute inflammatory arthritis, in particular a crystal-associated arthropathy or flares or acute forms of tendinitis and capsulitis by intra-articular injection into a joint of said pharmaceutical composition, wherein the microparticles have a mean particle size greater than 10 μm, wherein the time required to release 80% by weight of the colchicine in the microparticles is superior to 1 day and may reach 15 days, wherein the colchicine is present in a concentration ranging from 2.5 to 2500 μg per ml of suspension and wherein the pharmaceutical composition has a volume ranging from 0.1 ml to 5 ml.

Herein is further described a pharmaceutical composition under the form of a suspension comprising controlled release microparticles comprising colchicine, suitable for intra-articular injection into a joint, for use in the treatment of a crystal and non-crystal-associated acute inflammatory arthritis or acute forms of tendinitis and capsulitis, wherein the microparticles have a mean particle size greater than 10 μm, wherein the time required to release 80% by weight of the colchicine in the microparticles lies between 1 day and 15 days, wherein the colchicine is present in a concentration ranging from 0.05 to 5 mg per ml of suspension and wherein the pharmaceutical composition has a volume ranging from 0.1 ml to 5 ml.

According to a second main embodiment, herein is further provided a formulation under the form of a powder comprising controlled release microparticles comprising colchicine and a polymeric matrix, wherein the microparticles have a mean particle size equal or greater than 10 μm, and wherein the polymeric matrix comprises at least a poly(lactic-co-glycolic acid) copolymer, at least a poly(caprolactone) or at least a mixture of poly(lactic-co-glycolic acid) copolymer and poly(caprolactone), wherein the percentage weight ratio between the colchicine and the total weight of the microparticles ranges 0.1 to 35% by weight, in particular from 0.5 to 30% by weight, more particularly between 1 and 25% by weight, and even more particularly between 2 and 20% by weight.

Herein is further described a formulation under the form of a powder comprising controlled release microparticles comprising colchicine and a polymeric matrix, wherein the microparticles have a mean particle size equal or greater than 10 μm, and wherein the polymeric matrix comprises at least a poly(lactic-co-glycolic acid) copolymer, at least a poly(caprolactone) or at least a mixture of poly(lactic-co-glycolic acid) copolymer and poly(caprolactone), wherein the percentage weight ratio between the colchicine and total weight of the microparticles ranges from 1 to 35% by weight, in particular from 2 to 30% by weight, more particularly between 5 and 25% by weight, and even more particularly between 10 and 20% by weight.

According to a third main embodiment, herein is further provided a pharmaceutical composition comprising controlled release microparticles comprising colchicine, wherein the microparticles have a mean particle size greater than 10 μm, wherein said pharmaceutical composition presents an in vitro dissolution profile, wherein less than 25% of colchicine is released within 60 minutes, less than 50% of colchicine is released in 12 hours, wherein the colchicine is present in a concentration ranging from 2.5 to 2500 μg per ml of suspension and wherein the pharmaceutical composition has a volume ranging from 0.1 ml to 5 ml.

According to a fourth main embodiment, herein is further provided a formulation under the form of a powder comprising controlled release microparticles comprising colchicine and a polymeric matrix, wherein the microparticles have a mean particle size equal or greater than 10 μm, and wherein the polymeric matrix comprises at least a poly(lactic-co-glycolic acid) copolymer, at least a poly(caprolactone) or at least a mixture of poly(lactic-co-glycolic acid) copolymer and poly(caprolactone), wherein the percentage weight ratio between the colchicine and the total weight of the microparticles ranges 0.1 to 35% by weight, in particular from 0.5 to 30% by weight, more particularly between 1 and 25% by weight, and even more particularly between 2 and 20% by weight.

According to a fifth main embodiment, herein is further provided a pharmaceutical composition under the form of a sterile and injectable suspension suitable for an intra-articular injection, obtained by mixing a formulation under the form of a powder as described above, with an aqueous injection vehicle, wherein the pharmaceutical composition optionally comprises an excipient selected from a group consisting of a tonicity-enhancing agent, a wetting-agent, a viscosity-enhancing agent, a density-enhancing agent or a mixture thereof, and wherein said excipient may be present in the aqueous injection vehicle or in the powder.

The pharmaceutical composition suitable for an intra-articular injection as described herein after is effective in treating crystal-associated and non-crystal associated acute inflammatory arthritis, in particular effective in treating non-crystal associated acute arthritis, such as Rheumatoid arthritis, Ankylosing spondylitis, Juvenile arthritis, Psoriatic arthritis, Lupus or in treating crystal-associated arthropathies attacks or flares, such as gout, chondrocalcinosis, calcifying tendinitis and Milwaukee shoulder syndrome with minimal long-term side effects.

Said pharmaceutical composition suitable for an intra-articular injection is suitable for local or synovial administration by injection into a site at or near the site of pain.

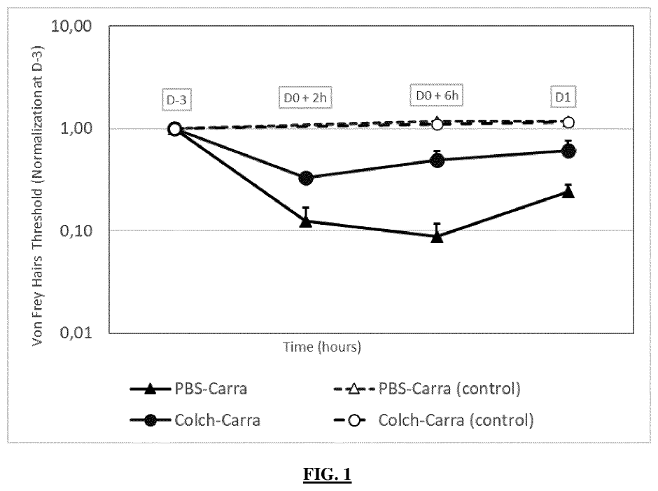

FIG. 1. represents the results of a in vivo studies demonstrating the efficacy in the treatment of pain of the suspension conform to the present invention as injected into ankle joint in a rat model.

### DETAILED DESCRIPTION OF THE INVENTION

As apparent from the BACKGROUND paragraph above, the prescription of colchicine is nowadays delicate as only oral compositions are available and high risk of toxicity have been observed, such as gastrointestinal symptoms and neuromuscular involvement. Risks linked to interaction with other active agents have further been reported. And again, toxicity constraints frankly have discouraged the local injection for decades.

Indeed, it has been established in the literature that effective local dose of 0.015 mg/kg to 0.030 mg/kg colchicine is recommended, that at 0.1 mg/kg the toxicity limit is reached, and that at 0.8 mg/kg lethal dose is reached (E. Niel et al, “Colchicine today”, Joint Bone Spine 73 (2006) 672-678). It is thus concluded that the narrow therapeutic margin for colchicine is a source of concern for prescribing physicians.

The inventors have found that compared to the treatment of oral colchicine or intra-articular corticosteroids in joint pain diseases, the intra-articular injection of a pharmaceutical composition under the form of a suspension for a controlled release of colchicine has at least equivalent and even higher efficiency, when the time required to release 80% by weight of the colchicine in the microparticles is greater than 1 day and may reach 15 days and when the colchicine is present in a concentration ranging from 2.5 to 2500 μg per ml of suspension and wherein the pharmaceutical composition has a volume ranging from 0.1 ml to 5 ml.

The diseases for which the use of said pharmaceutical composition is dedicated are very intense. Thus, having treatments perfectly adapted for achieving the most appropriate dosage is crucial.

The use or method of treatment according to the present invention also has the advantage of reducing the significant toxicity risks, linked to drug interaction with colchicine when administered via a systemic route, and in particular in the elderly, or patients with kidney or liver failure as explained herein after.

In comparison to intra-articular corticosteroids, this treatment also has the advantage to avoid systemic effects such as Cushing's syndrome, hyperglycemia, hypertension, decreased bone density or even the risk of infections. In addition, it decreases the risk of deleterious effects on local tissue as damage to articular cartilage and tendons.

### Definitions

As used herein, the term “patient” refers to either an animal, such as a valuable animal for breeding, company or preservation purposes, or preferably a human or a human child, which is afflicted with, or has the potential to be afflicted with one or more diseases and conditions described herein.

In particular, as used in the present application, the term “patient” refers to a mammal, including a non-human mammal such as a rodent, cat, dog, or primate, or a human; preferably said subject is a human and also extends to birds.

The identification of those patients who are in need of treatment of herein-described diseases and conditions is well within the ability and knowledge of one skilled in the art. A veterinarian or a physician skilled in the art can readily identify, by the use of clinical tests, physical examination, medical/family history or biological and diagnostic tests, those patients who are in need of such treatment.

In the context of the invention, the term “treating” or “treatment”, as used herein, means reversing, alleviating, inhibiting the progress of non-crystal associated acute inflammatory arthritis, such as Rheumatoid arthritis, Ankylosing spondylitis, Juvenile arthritis, Psoriatic arthritis, Lupus or in treating crystal-associated acute inflammatory arthritis, in particular crystal-associated arthropathies attacks or flares, such as gout, chondrocalcinosis, calcifying tendinitis and Milwaukee shoulder syndrome, and acute forms of tendinitis and capsulitis.

As used herein, an “effective amount” refers to an amount of a compound of the present invention which is effective in reducing, eliminating, treating or controlling the symptoms of the herein-described diseases and conditions, i.e. crystal-associated and non-crystal associated acute inflammatory arthritis, in particular non-crystal associated acute arthritis, such as Rheumatoid arthritis, Ankylosing spondylitis, Juvenile arthritis, Psoriatic arthritis, Lupus or in treating and crystal-associated arthropathies attacks or flares, such as gout, chondrocalcinosis, calcifying tendinitis and Milwaukee shoulder syndrome, and acute forms of tendinitis and capsulitis. The term “controlling” is intended to refer to all processes wherein there may be a slowing, interrupting, arresting, or stopping of the progression of the diseases and conditions described herein, but does not necessarily indicate a total elimination of all diseases and condition symptoms.

The term “treatment-effective amount” refers to a concentration of compound that is effective in treating crystal-associated and non-crystal associated acute inflammatory arthritis, in particular non-crystal associated acute arthritis, such as Rheumatoid arthritis, Ankylosing spondylitis, Juvenile arthritis, Psoriatic arthritis, Lupus or in treating crystal-associated arthropathies attacks or flares, e.g. leads to a reduction in inflammatory pain, following examination when administered after initiation of the inflammatory pain has occurred.

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

The term “pharmaceutically acceptable carrier, adjuvant, or vehicle” may refer to any pharmaceutically acceptable excipient, such as a non-toxic carrier, adjuvant, or vehicle that does not destroy the pharmacological activity of the compound with which it is formulated.

Within the framework of the present invention, the term “painful site” or “site of the pain” means the joint area causing pain where the product can be injected. It can be the knee joint, the hip joint, metacarpophalangeal joints, the shoulder joint, the wrist joint, the elbow joint, the metatarsophalangeal joint, the ankle joint, the vertebral joint and the midfoot joints.

The terms “controlled release composition” or “controlled release microparticle” mean the composition or the microparticle allow a release into the body of a specified amount over a specified period of time of an active ingredient, namely a specific pharmacokinetic profile. The term “controlled release” encompasses all the type of releases that are modified in comparison to an immediate release. In other words, the term “controlled release” is equivalent to “modified release” and encloses extended releases as defined herein after as well as delayed release and pulse releases.

The terms “extended release”, “prolonged release” and “sustained release” are considered equivalent in the framework of the present invention. This type of release means that the release is prolonged over time in comparison to an immediate release, i.e. it means that the active ingredient is released slowly over time, allowing a less frequent intake of the drug for the patient. In other words, the active ingredient is progressively released over a specific period of time, generally aiming at less side effects as well by decreasing the maximal concentration.

The term “drug loading content” means the mass ratio of the drug in the microparticles to the mass of microparticles.

The term “mean particle size” or D50 means the particle diameter in microns that splits the particle volume distribution with half above and half below this diameter.

The term “D10=x μm” as mainly used in the example means that 10% of the particles have a size of x μm or less.

The term “D90=y μm” as mainly used in the example means that 90% of the particles have a size of y μm or less.

A controlled or modified release may of course result from the combination of an immediate release and a controlled release.

### Colchicine

Colchicine is an alkaloid extracted from the corm of the meadow saffron or autumn crocus (Colchicum autumnale). Its IUPAC name is N-[(7S)-1,2,3,10-tetramethoxy-9-oxo-6,7-dihydro-5H-benzo[a]heptalen-7-yl]acetamide. (CAS 64-86-8)

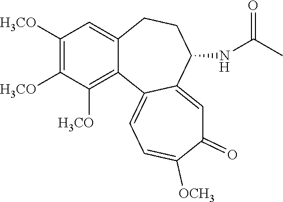 [SMILES: COc1cc2c(c(OC)c1OC)-c1ccc(OC)c(=O)cc1[C@@H](NC(C)=O)CC2]

Colchicine may exist in racemic of enantiomeric form. All said forms are encompassed within the scope of the invention.

Moreover, it may exist in various crystalline forms depending on the solvent used for obtaining them. Chloroform, dichloromethane, ethyl acetate, benzene or any other suitable solvent may be cited among possible solvents. All such crystalline forms also form part of the present invention.

It is mainly known as gout suppressant and as an anti-inflammatory agent via its tubulin interaction activity. It is administered by the oral route although intravenous administration was also attempted and then abandoned for excessive toxicity reasons.

### Therapeutic Use and Methods

In one embodiment, the pharmaceutical composition suitable for an intra-articular injection as described herein after delivers colchicine in a dose and in a controlled release manner such that the concentration level of colchicine at the treated articular site is above the systemic dosage classically obtained after an oral administration, i.e. above 5 nM (2 ng/mL), and the systemic concentration level stays below the value of 15 nM (6 ng/mL). (Terkeltaub, R. A. et al. High versus low dosing of oral colchicine for early acute gout flare: Twenty-four-hour outcome of the first multicenter, randomized, double-blind, placebo-controlled, parallel-group, dose-comparison colchicine study. Arthritis Rheum. 62, 1060-1068 (2010)).

According to one embodiment, the pharmaceutical composition of the invention is further characterized in that it is effective to maintain systemic concentration of colchicine after 12 hours lower than 5 ng/ml, particularly lower than 2 ng/ml and more particularly lower than 1 ng/ml and to maintain synovial concentration of colchicine greater than 2 ng/ml, in particular comprised between 2 and 500 ng/ml, more particularly comprised between 2 and 250 ng/ml, more particularly comprised between 2 and 200 ng/ml, for example between 2 and 100 ng/ml, and even more particularly between 2 and 50 ng/ml.

The particular release profile allowing a tolerable systemic concentration of colchicine and at the same time a local concentration of colchicine suitable for having an appropriate therapeutic effect, which represent the very core of the invention, has never been obtained before and is in particular illustrated in example 7 herein after in a in vivo model in the rabbit. These results combined with scaling from body weight to classical allometric models such ones proposed by Health Authorities for predicting human dosage from rabbit dosage allow to support the resolution of the technical problem the man skilled in the art was faced to in dosing colchicine due to the exceptionally narrow therapeutic index as explained above.

It comes out that through a particular ratio between the local or synovial concentration of colchicine and systemic concentration of colchicine obtained with the pharmaceutical composition according to the invention, i.e. with a particular in vitro dissolution rate and/or in vivo release rate, the inventors have discovered a surprisingly well adapted mean for releasing the patients while avoiding side effects. In other words, said parameters have been seriously evaluated and defined before the controlled formulation conform to the present invention be successfully devised.

According to one particular embodiment, the ratio between the local or synovial concentration of colchicine and systemic concentration of colchicine obtained after injection into a joint of a pharmaceutical composition according to the present invention may range between 100 and 1000.

For determining said local and systemic concentration of colchicine, the man skilled in the art may use any known method. For example, a LC-MS analysis may be used.

Illustration of said particular control of the release of colchicine as achieved in the framework of the present invention may also be illustrated and characterized via in vitro dissolution profiles as illustrated in examples 1, 2, 3 and also performed in example 8.

According to one embodiment, the dissolution medium that may be used for performing such dissolution test may be a phosphate buffer (pH 7.4).

According to one embodiment, the rotation speed may range from 50 to 200, in particular from 60 to 100 rpm. This agitation may be obtained thanks to shaking incubator, in particular with horizontal motion.

According to one embodiment, the dissolution is measured for example by UV analysis using a UV spectrophotometer, for example at 350 nm, at different intervals, for example at 1 day, 2 days, 3 days, 5 days, 7 days, 8 days, 9 days, 10 days and 13 days.

By way of example, a dissolution test may be performed according to the herein after detailed method:

Aliquots of 10-30 mg colchicine microparticles are suspended in 50 mL of phosphate buffer (50 mM, pH 7.4) containing 0.02% sodium azide maintained at 37° C. in a horizontal shaking incubator set at 80 rpm, for example a 3033 model commercialized by GFL. At different intervals, 3 mL of the media are removed and replaced with an equivalent amount of fresh media, and analyzed by UV at 350 nm.

As above mentioned, the controlled release form is administered locally to treat pain and inflammation in the joints. Local administration of the formulation may occur by injection into the intra-articular space or peri-articular space at or near the site of a patient's pain, including the metatarsophalangeal joints, the metacarpophalangeal joints, the knee joint, the shoulder joint, the wrist joint, the elbow joint, the ankle joint, the hip joint, a vertebral joint and the midfoot joints. The midfoot is the anterior part of the foot that sits between the hindfoot and forefoot. It is composed of the cuboid, navicular and three cuneiform bones. The mid-tarsal and the tarsometatarsal joints join the midfoot to the hindfoot and the forefoot respectively. The midfoot is a complex and composite area where observed oedema inflammation is generally diffuse.

In one embodiment, the present invention is dedicated to the treatment of pain in joints, in particular crystal-associated and non-crystal associated acute inflammatory arthritis, more particularly selected from non-crystal associated acute arthritis, such as Rheumatoid arthritis, Ankylosing spondylitis, Juvenile arthritis, Psoriatic arthritis, Lupus or in treating and crystal-associated arthropathies attacks or flares, such as gout, chondrocalcinosis, calcifying tendinitis and Milwaukee shoulder syndrome, and acute forms of tendinitis and capsulitis.

As illustrated in example 10, pain has been measured in a carrageenan-induced inflammation rat model following the injection of a pharmaceutical composition according to the present invention into ankle joint in a rat model, demonstrating that a significant decrease of pain from 2 hours following injection up to at least 24 hours.

In one embodiment, the composition suitable for an intra-articular injection, is dedicated to the treatment of gout attack.

In one embodiment, the composition suitable for an intra-articular injection is dedicated to the treatment of chondrocalcinosis.

In one embodiment, the composition suitable for an intra-articular injection is dedicated to the treatment of acute and chronic calcifying tendinitis.

In one embodiment, the composition suitable for an intra-articular injection is dedicated to the treatment of Milwaukee shoulder syndrome.

In one embodiment, composition suitable for an intra-articular injection is dedicated to the treatment of tendinitis.

In one embodiment, composition suitable for an intra-articular injection is dedicated to the treatment of capsulitis.

The administration may be performed by a single injection or as sequential injections provided that the time required to release 80% by weight of the colchicine in the microparticles is greater than 1 day and may reach 15 days, in particular lies between 1.5 days and 15 days, the colchicine being present in a concentration ranging from 2.5 to 2500 μg per ml of suspension and the pharmaceutical composition having a volume ranging from 0.1 ml to 5 ml. In other words, the single or sequential injections, preferably the single injection, provides locally a colchicine weight ranging between 0.25 and 12500 pgof colchicine.

In one embodiment, the injection is performed a plurality of times (two times, three times, four times or more). In this embodiment, each subsequent time the injection is performed, it is performed after a suitable period has lapsed since the preceding time the injection was performed. This suitable time can be, for example, two weeks, one month, two months, three months, four months, five months, six months, one year, or longer.

According to a particular embodiment, the colchicine is present in a concentration ranging from 0.1 to 4 mg per ml of suspension, in particular from 0.1 to 2.5 mg per ml of suspension, in particular from 0.25 to 2.5 mg per ml of suspension, and more particularly from 0.5 to 1.5 mg per ml of suspension.

According to another particular embodiment, the colchicine is present in a concentration ranging from 5 to 2000 μg per ml of suspension, in particular from 5 to 1500 μg per ml of suspension, in particular from 5 to 1000 μg per ml of suspension, and more particularly from 5 to 500 μg per ml of suspension.

In a particular embodiment, the same pharmaceutical composition under the form of a suspension may be used for intra-articular injection to any painful joint. The amount of pharmaceutical composition to be injected may however be adapted to the targeted joint. This is one of the advantages of the invention, which may with a single concentration offer the adapted amount of colchicine for each site of application by adapting the corresponding needed volume.

In one embodiment, injections may be performed simultaneously in two or more different painful joints.

For example, the volume of pharmaceutical composition under the form of a suspension according to the present invention that may be injected to the shoulder may vary between 2 and 5 ml.

The volume of pharmaceutical composition under the form of a suspension according to the present invention that may be injected to the metatarsophalangeal joints, for example the toes, may vary between 0.1 and 0.5 ml, typically is 0.25 ml.

The volume of pharmaceutical composition under the form of a suspension according to the present invention that may be injected to the metacarpophalangeal joints, for example the fingers, may vary between 0.1 and 0.5 ml, typically is 0.25 ml.

The volume of pharmaceutical composition under the form of a suspension according to the present invention that may be injected to a vertebral joint may vary between 1 and 2 ml.

The volume of pharmaceutical composition under the form of a suspension according to the present invention that may be injected to the midfoot joints may vary between 0.1 and 1 ml.

The volume of pharmaceutical composition under the form of a suspension according to the present invention that may be injected to the knee, may vary between 2 and 5 ml.

The volume of pharmaceutical composition under the form of a suspension according to the present invention that may be injected to the wrist may vary between 0.5 and 1 ml.

The volume of pharmaceutical composition under the form of a suspension according to the present invention that may be injected to the elbow may vary between 2 and 5 ml.

The volume of pharmaceutical composition under the form of a suspension according to the present invention that may be injected to the ankle may vary between 1 and 3 ml.

The volume of pharmaceutical composition under the form of a suspension according to the present invention that may be injected to the hip may vary between 2 and 5 ml.

In one embodiment, the pharmaceutical composition for use according to the present invention may be characterized by its volume, which may be adapted to the joint, and namely may range from 2 to 5 ml for the shoulder, from 1 to 2 ml for a vertebral joint, from 0.1 to 1 ml for the midfoot joints, from 0.1 to 0.5 ml for the metatarsophalangeal joints, from 0.1 to 0.5 ml for the metacarpophalangeal joints, from 2 to 5 ml for the knee joint, from 0.5 to 1 ml for the wrist joint, from 2 to 5 ml for the elbow joint, from 1 to 3 ml for the ankle joint and from 2 to 5 ml for the hip.

It should also be understood that a specific dosage and treatment regimen for any particular patient will depend upon a variety of factors, including the age, body weight, general health, sex, diet, time of administration, drug combination, and the judgment of the treating physician and the severity of the particular disease being treated.

In one embodiment, the controlled release is suitable for reaching an articular colchicine maximum concentration (Cmax) ranging from 5 nM (2 ng/mL) to 5 μM (2 μg/mL).

According to a preferred embodiment, the injection of the pharmaceutical composition of the present invention allows reaching a systemic concentration of colchicine which does not exceed 5 ng/ml, in particular not exceeding 2 ng/ml, and more particularly not exceeding 1 ng/ml 12 hours after the intra-articular injection.

In addition, the injection of the controlled release pharmaceutical composition according to the invention can be able to achieve a synovial concentration of colchicine greater than 2 ng/ml, in particular comprised between 2 and 500 ng/ml, more particularly comprised between 2 and 250 ng/ml, more particularly comprised between 2 and 200 ng/ml, for example between 2 and 100 ng/ml, and even more particularly between 2 and 50 ng/ml.

The requested dose to be injected into the painful joint can depend on the type of painful joint, in particular depending on its size. Table 1 as follows gathers typical dosage that may be implemented in the framework of the present invention.

TABLE 1

| | | | Drug | |
|---|---|---|---|---|
| | | Volume | concentration | Dose range |
| | Joint | (ml) | (μg/ml) | (μg) |
| Small joints | metatar- | 0.1-0.5 | 2.5-2500 | 0.25-1250 |
| | sophalangeal | | | |
| | metacar- | 0.1-0.5 | 2.5-2500 | 0.25-1250 |
| | pophalangeal | | | |
| | wrist | 0.5-1 | 2.5-2500 | 1.25-2500 |
| | midfoot | 0.1-1 | 2.5-2500 | 0.25-2500 |
| Medium/large | vertebral | 1-2 | 2.5-2500 | 2.5-5000 |
| joints | ankle | 1-3 | 2.5-2500 | 2.5-7500 |
| | knee | 2-5 | 2.5-2500 | 5-12500 |
| | hip | 2-5 | 2.5-2500 | 5-12500 |
| | shoulder | 2-5 | 2.5-2500 | 5-12500 |
| | elbow | 2-5 | 2.5-2500 | 5-12500 |

In one embodiment, the pharmaceutical composition as used in the present invention, which is in particular administered in one intra-articular injection, and which is dedicated to the treatment of the acute phase of crystal-associated inflammatory arthritis or non-crystal associated inflammatory arthritis or acute forms of tendinitis and capsulitis, comprises between 0.25 to 12500 μg of colchicine per injection for a small joint selected from metatarsophalangeal joint or metacarpophalangeal joint.

In another embodiment, the pharmaceutical composition as used in the present invention, which is in particular administered in one intra-articular injection, and which is dedicated to the treatment of the acute phase of crystal-associated inflammatory arthritis or non-crystal associated inflammatory arthritis or acute forms of tendinitis and capsulitis, comprises between 1.25 and 2500 μg of colchicine per injection for a wrist joint.

In another embodiment, the pharmaceutical composition as used in the present invention, which is in particular administered in one intra-articular injection, and which is dedicated to the treatment of the acute phase of the crystal-associated inflammatory arthritis or non-crystal associated inflammatory arthritis or acute forms of tendinitis and capsulitis, comprises between 0.25 and 2500 μg per injection for midfoot joints.

In another embodiment, the pharmaceutical composition as used in the present invention, which is in particular administered in one intra-articular injection, and which is dedicated to the treatment of the acute phase of the crystal-associated inflammatory arthritis or non-crystal associated inflammatory arthritis or acute forms of tendinitis and capsulitis, comprises between 2.5 and 7500 μg per injection for an ankle or 2.5 and 5000 μg per injection vertebral joint.

In another embodiment, the pharmaceutical composition as used in the present invention, which is in particular administered in one intra-articular injection, and which is dedicated to the treatment of the acute phase of the crystal-associated inflammatory arthritis or non-crystal associated inflammatory arthritis or acute forms of tendinitis and capsulitis, comprises between 5 and 12500 μg per injection for a joint selected from a knee joint, a hip joint, a shoulder joint and an elbow joint.

According to one embodiment, the pharmaceutical composition as used in the present invention, which is in particular administered in one intra-articular injection, and which is dedicated to the treatment of the acute phase of the crystal-associated inflammatory arthritis or non-crystal associated inflammatory arthritis or acute forms of tendinitis and capsulitis, comprises between 0.25 to 12500 μg, in particular 0.25 and 10000 μg of colchicine, more particularly between 0.25 and 7500 μg, for example between 0.25 and 500 μg. In one embodiment, the pharmaceutical composition as used in the present invention may for example comprise between 0.5 and 10000 μg of colchicine, in particular between 0.5 and 7500 μg of colchicine, more particularly between 0.5 and 500 μg of colchicine. In one embodiment, the pharmaceutical composition as used in the present invention may for example comprise between 1 and 10000 μg of colchicine, in particular between 1 and 7500 μg of colchicine, more particularly between 1 and 5000 μg of colchicine. In one embodiment, the pharmaceutical composition as used in the present invention may for example comprise between 2 and 10000 μg of colchicine, in particular between 2 and 7500 μg of colchicine.

In one embodiment, the pharmaceutical composition may be suited for maintaining a therapeutically acceptable concentration of colchicine in the painful joint during a period superior to one day to 9 days, a period superior to one day to 8 days, a period superior to one day to 7 days, a period superior to one day to 6 days, and for example during 5 days, 6 days, 7 days, 8 days, 9 days or 10 days, depending on the nature of the pathology to be treated, more particularly during 5 days.

In one embodiment, the pharmaceutical composition for use according to the invention, is characterized by the fact that the time required to release 80% by weight of the colchicine in the microparticles is superior to 1 day, for example 1.5 day, and may reach 10 days, is superior to 1 day, for example 1.5 day, and may reach 7 days, is superior to 1 day, for example 1.5 day, and may reach 5 days, and for example is superior to 1 day, for example 1.5 day, 2 days, 3 days, 4 days, 5 days, 6 days, 7 days, 8 days, 9 days or 10 days, more particularly is superior to 1 day, for example 1.5 day, and may reach 7 days, or is comprised between 2 days and 15 days, between 2 days and 10 days and between 2 days and 7 days.

In one embodiment, the pharmaceutical composition as used in the present invention may be administered during 5 to 10 days and it comprises between 1 to 7.5 mg of colchicine, between 2 to 6 mg of colchicine or between 3 to 5 mg of colchicine.

In one embodiment, the pharmaceutical composition as used in the present invention may be administered during 6 to 9 days and it comprises between 2 to 7.5 mg or colchicine, in particular between 3 to 5 mg of colchicine.

It is apparent that the dosage of colchicine may be reduced in some embodiments by 10%, 20%, 30%, 40% and even more for the treatment of one acute phase of crystal-associated inflammatory arthritis or non-crystal associated inflammatory arthritis or acute forms of tendinitis and capsulitis.

According to one particular embodiment, the treatment may be renewed when another acute flare arises under the same conditions and posology.

According to one embodiment, and more particularly for the treatment of acute arthritis associated with the presence of microcrystals or non-crystal associated inflammatory arthritis, and more particularly gout attacks, chondrocalcinoses and acute calcifying tendinitis, the pharmaceutical composition may be suited for providing an initial articular colchicine concentration.

The pharmaceutical composition may or may not exhibit a burst release. In the framework of the present invention a “burst release” means that an initial bolus of drug is released before the release rate reaches a stable profile, immediately upon placement in the release medium, i.e. in the present invention immediately after the injection into the articulation.

In any case, the length of burst release, may be between 0 and 1 day, for example between the beginning of day 1 through the end of day 1, and in particular is between 0 and 6 hours, between 0 and 3 hours, and even more particularly is between 0 and 2 hours.

Thus, when a burst release occurs, upon administration, the pharmaceutical composition according to the present invention may provide an initial release of colchicine at the site of administration, for example, in the intraarticular space and/or peri-articular space. Once the initial release of colchicine has subsided, the controlled release of the colchicine microparticle formulation continues to provide therapeutic (e.g., intra-articular and/or peri-articular) concentrations of colchicine to combat inflammation and/or pain for an additional period of therapy following administration.

In a particular embodiment, the pharmaceutical composition for use according to the present invention does not exhibit an important burst release. According to said embodiment, less than 50% by weight of colchicine is released at 12 hours after the administration. This is in particular a distinguishing characteristic with respect to Aboumanei et al, as discussed in the preamble.

According to another aspect, the invention relates to a method for the treatment of crystal-associated and non-crystal associated acute inflammatory arthritis, in particular a non-crystal associated acute arthritis, such as Rheumatoid arthritis, Ankylosing spondylitis, Juvenile arthritis, Psoriatic arthritis, Lupus or a crystal-associated arthropathy attacks or flares or acute forms of tendinitis and capsulitis comprising at least the administering by injection to a painful joint to a patient in need thereof of a pharmaceutical composition comprising controlled release microparticles comprising an effective amount of colchicine, suitable for intra-articular injection into a joint, wherein the microparticles have a mean particle size greater than 10 μm, wherein the time required to release 80% by weight of the colchicine in the microparticles is greater than 1 day and may reach 15 days, in particular is comprised between 1.5 days and 15 days, wherein the colchicine is present in a concentration ranging from 2.5 to 2500 μg per ml of suspension and wherein the pharmaceutical composition has a volume ranging from 0.1 ml to 5 ml.

Still according to another aspect, the invention relates to a method for the treatment of crystal-associated and non-crystal associated acute inflammatory arthritis, in particular a non-crystal associated acute arthritis, such as Rheumatoid arthritis, Ankylosing spondylitis, Juvenile arthritis, Psoriatic arthritis, Lupus or a crystal-associated arthropathy attacks or flares or acute forms of tendinitis and capsulitis comprising at least the administration by injection into the joints of a patient in need thereof of at least an efficient amount of colchicine for a controlled release.

Herein is further provided a method for the treatment of crystal-associated and non-crystal associated acute inflammatory arthritis, in particular of crystal-associated arthropathy attacks or flares or acute forms of tendinitis and capsulitis comprising at least the steps of:
preparing and/or providing a pharmaceutical composition under the form of a sterile and injectable suspension by mixing a formulation under the form of a powder as described herein after, with an aqueous injection vehicle, wherein the pharmaceutical composition optionally comprises an excipient selected from a group consisting of a tonicity-enhancing agent, a wetting-agent, a viscosity-enhancing agent, a density-enhancing agent or a mixture thereof, and wherein said excipient may be present in the aqueous injection vehicle or in the powder, and
injecting said pharmaceutical composition into a painful joint of a patient in need thereof, wherein the volume to be injected is adapted to the injection site.

Herein is further provided a kit or article of manufacture comprising, in separate compartments (i) an aqueous injection vehicle and (ii) a powder as described herein after, wherein excipient selected from a group consisting of a tonicity-enhancing agent, a wetting-agent, a viscosity-enhancing agent, a density-enhancing agent or a mixture thereof may be present in the aqueous injection vehicle or in the powder, for preparing a pharmaceutical composition suitable for an intra-articular injection.

In said embodiment, the kit or article of manufacture may be under the form of a prefilled syringe or medical device, for example comprising a dual chamber. In said embodiment, the two compartments or dual chamber may allow the mixture of the aqueous injection vehicle and the powder as described herein after by all means known to the man skilled in the art. Still in said embodiment, said means may take the form of a pierceable membrane or destructible diaphragm, for example through a pressure the user may exert.

In said embodiment, the kit or article of manufacture may additionally contain a label instructing the user to introduce the obtained pharmaceutical composition into a subject's joint.

The present invention may be particularly suitable for treating patients having renal and/or hepatic impairment or patients being simultaneously treated with drugs. Indeed, for patients having renal and/or hepatic impairment, adjustments of the recommended oral dosage of colchicine, in particular in the treatment of gout flares; are required of adaptation of the administration scheme. Moreover, some drugs have been identified in the past with oral colchicine treatments as generating undesirable side effects and even fatal drug interactions, drastically modifying the safety profile and necessitating oral colchicine dosage adjustments which may for example require a reduction by two to three times of the oral colchicine dosage.

Thus, in one embodiment, the pharmaceutical composition conform to the present invention may be administered to patients having renal and/or hepatic impairment. In particular, colchicine plasmatic level is deemed to rise to toxicity threshold when intrinsic factor renal or hepatic impairment achieve Severe grade.

Patients having renal impairment may be identified and classified by techniques known to the physicians such as via the Creatinine Clearance method or via the Glomerular Filtration Rate (GFR) method. Said methods are in particular detailed in Worboys P D, Wong S L, Barriere S L. Pharmacokinetics of intravenous telavancin in healthy subjects with varying degrees of renal impairment. Eur J Clin Pharmacol. 2015 June; 71(6):707-714. The use and method according to the present invention may for example be implemented to treat patients having all grades of renal impairment such as grades 1 to 5 as set by the GFR method. The use and method according to the present invention may be more particularly suited for patients having high grades of renal impairment, such as grades 3 to 5 as set by the GFR method Patients having hepatic impairment may be identified and classified by techniques known to the physicians such as via the Score of Child-pugh method or via the MELD score. Said methods are in particular detailed in Tsoris A, Marlar C A. Use Of The Child Pugh Score In Liver Disease. 2021 Mar. 22. In: StatPearls [Internet]. Treasure Island (FL): StatPearls Publishing; 2022 January-. PMID: 31194448 or on internet under the following links: https://liverfellow.org/post/meld-score-part1 and https://liverfellow.org/Rost/meld score-part2. The use and method according to the present invention may for example be implemented to treat patients having all scores, rates and/or classification, for example mild, moderate of severe hepatic impairment as set by the Child-Pugh method. The use and method according to the present invention may be more particularly suited for patients having high scores, rates and/or classification of severe hepatic impairment as set by the Child-Pugh method such as moderate and severe hepatic impairment.

Thus, due to the colchicine systemic plasmatic level being maintained under the threshold leading to toxicity in the framework of the present invention, no colchicine dosage adjustment is required even for treating patients with intrinsic factor impacts, such as renal and/or hepatic impairment that currently require colchicine dosage adjustments when orally administered.

Thus, in one embodiment, the pharmaceutical composition for use according to the present invention may further be characterized by the fact that the patient to which said pharmaceutical composition is administered have renal and/or hepatic impairment.

In one other embodiment, further provided a method for the treatment of crystal-associated and non-crystal associated acute inflammatory arthritis, in particular a non-crystal associated acute arthritis, such as Rheumatoid arthritis, Ankylosing spondylitis, Juvenile arthritis, Psoriatic arthritis, Lupus or a crystal-associated arthropathy attacks or flares or acute forms of tendinitis and capsulitis as defined herein above to a patient having renal and/or hepatic impairment, in particular wherein the dosage of colchicine is not reduced in response to such impairments.

In one embodiment, the pharmaceutical composition conform to the present invention may be co-administered with a wide range of drugs as detailed herein after, in particular showing improved safety profiles in comparison to the classical treatment with oral colchicine when co-administered with such drugs.

As an example of drug interaction that the present invention allows to avoid are the interactions with drugs that have been clearly identified in the framework of treatments with oral colchicine and that are well known to the physicians. In a situation of potential drug interaction, the classical way to proceed consists in adjusting, namely lowering the dosage of oral colchicine, depending on the severity of the interaction, according to well-known recommendations.

Such drug drug interactions (DDI) may be explained by the fact that some drugs inhibit P-gp and/or CYP3A4, generating peaks of excessive plasmatic concentration of colchicine, and in particular peaks above 6 ng/mL of colchicine, that must be avoided.

One advantage of the present invention relies in the fact that excessive plasmatic concentration is avoided and allows co-administration of drugs without adjusting the colchicine dosage it must be done when orally administered.

Thus, in one embodiment, the pharmaceutical composition for use according to the present invention may further be characterized by the fact that the patient to which said pharmaceutical composition is administered may simultaneously, separately or sequentially be treated by the following active ingredients: atazanavir, clarithromycin, darunavir, ritonavir, indinavir, itraconazole, ketoconazole, lopinavir, ritonavir, nefazodone, nelfinavir, ritonavir, saquinavir, telithromycin, tipranavir, amprenavir, aprepitant, diltiazem, erythromycin, fluconazole, fosamprenavir, verapamil, cyclosporine and ranolazine.

Herein is thus provided the pharmaceutical composition for use as defined above wherein it is for use in the treatment of crystal-associated and non-crystal associated acute inflammatory arthritis, in particular a crystal-associated arthropathy or flares or acute forms of tendinitis and capsulitis in a patient being simultaneously, separately or sequentially treated by at least one of the active ingredient selected from: atazanavir, clarithromycin, darunavir, ritonavir, indinavir, itraconazole, ketoconazole, lopinavir, ritonavir, nefazodone, nelfinavir, ritonavir, saquinavir, telithromycin, tipranavir, amprenavir, aprepitant, diltiazem, erythromycin, fluconazole, fosamprenavir, verapamil, cyclosporine and ranolazine.

Herein is thus further provided a method for the treatment of crystal-associated and non-crystal associated acute inflammatory arthritis, in particular a non-crystal associated acute arthritis, such as Rheumatoid arthritis, Ankylosing spondylitis, Juvenile arthritis, Psoriatic arthritis, Lupus or a crystal-associated arthropathy attacks or flares or acute forms of tendinitis and capsulitis as defined herein above to a patient being simultaneously, separately or sequentially treated by at least one active ingredient selected from: atazanavir, clarithromycin, darunavir, ritonavir, indinavir, itraconazole, ketoconazole, lopinavir, ritonavir, nefazodone, nelfinavir, ritonavir, saquinavir, telithromycin, tipranavir, amprenavir, aprepitant, diltiazem, erythromycin, fluconazole, fosamprenavir, verapamil, cyclosporine and ranolazine, in particular wherein the dosage of colchicine is not reduced in response to the concomitant administration of such drug.

### Controlled Release Microparticles Comprising Colchicine

The controlled release microparticles comprising colchicine may take the form of various microparticles, such as (i) microparticles comprising a polymeric matrix or (ii) multivesicular liposomes.

According to one embodiment, the microparticles have a mean particle size lower than 100 μm, in particular lower than 80 μm, and more particularly lower than 50 μm, for example between 10 and 50 μm, between 10 and 40 μm, or between 15 and 40 μm.

According to one embodiment, the microparticles have a particle size distribution having a D10 value of not more than 50 μm, in particular of not more than 40 μm, and for example from 5 μm to 40 μm.

According to one embodiment, the microparticles have a particle size distribution having a D90 value of not more than 150 μm, in particular of not more than 100 μm, and for example from 20 μm to 100 μm.

It is understood that these ranges refer to the mean size of all microparticles in a given population. The size of any given individual microparticle could be within a standard deviation above or below the mean size.

Within the context of the present invention, a “microparticle” means a particle of any shape and made of any material, in particular suitable for the injection into the human body within a pharmaceutical composition, and in particular into the articulations or joints, having a mean particle size greater than 10 μm and less than 100 μm.

The microparticles suitable for the pharmaceutical composition used according to the present invention may be selected form various types of microparticles such as microspheres, microparticle matrices, microsphere matrices, microcapsules, rods, wafers, pills, fibers and pellets.

### (i) Microparticles Comprising a Polymeric Matrix

The polymeric matrix may be chosen from various polymers suitable for obtaining the controlled release microparticles. Such polymeric matrix is nontoxic to the human body.

According to one embodiment, the polymer forming the polymeric matrix is biodegradable.

Within the context of the present invention, a “biodegradable” material, refers to a material which degrades enzymatically or hydrolytically and for which there is proof that the degradation products are integrated into biomass and/or eliminated from the organism by metabolism or renal filtration.

The nontoxic and biodegradable polymer may be natural or synthetic.

According to one embodiment, the polymeric matrix of the microparticles used in the pharmaceutical composition used according to the present invention comprises at least a poly(lactic-co-glycolic acid) copolymer or PLGA.

According to said embodiment, the polymeric matrix may comprise PLGA in an amount of greater than 70% by weight, with respect to the total weight of the polymeric matrix, in particular greater than 80% by weight, and even more particularly greater than 90% by weight.

According to another embodiment, the polymeric matrix of the microparticles used in the pharmaceutical composition used according to the present invention comprises at least a poly(caprolactone) (PCL).

Suitable polymers are not limited to those commercially available and known as RESOMER (Evonik Industries AG, Germany), LACTEL (Durect, USA), PURASORB (Corbion NV, The Netherlands), Viatel (Ashland, USA).

Examples of suitable polymers are listed in Table 2.

TABLE 2
Examples of suitable polymers

| | | End | |
|---|---|---|---|
| Product name | Polymer | group | Producer |
| Resomer | Poly(D,L-lactide-co-glycolide) | acid | Evonik |
| RG 502H | 50:50 | | |
| Resomer | Poly(D,L-lactide-co-glycolide) | ester | Evonik |
| RG 502 | 50:50 | | |
| Resomer | Poly(D,L-lactide-co-glycolide) | acid | Evonik |
| RG 503H | 50:50 | | |
| Resomer | Poly(D,L-lactide-co-glycolide) | ester | Evonik |
| RG 503 | 50:50 | | |
| Lactel | Poly(D,L-lactide-co-glycolide) | acid | Durect |
| B6013-1 | 50:50 | | |
| Lactel | Poly(D,L-lactide-co-glycolide) | ester | Durect |
| B6017-1 | 50:50 | | |
| Lactel | Poly(D,L-lactide-co-glycolide) | ester | Durect |
| B6010-1 | 50:50 | | |
| Purasorb | Poly(D,L-lactide-co-glycolide) | acid | Corbion |
| PDLG | 50:50 | | |
| 5002A | | | |
| Purasorb | Poly(D,L-lactide-co-glycolide) | ester | Corbion |
| PDLG | 50:50 | | |
| 5002 | | | |
| Purasorb | Poly(D,L-lactide-co-glycolide) | acid | Corbion |
| PDLG | 50:50 | | |
| 5004A | | | |
| Purasorb | Poly(D,L-lactide-co-glycolide) | ester | Corbion |
| PDLG | 50:50 | | |
| 5004 | | | |
| Viatel | Poly(D,L-lactide-co-glycolide) | acid/ester | Ashland |
| | 50:50 | | |

According to said embodiment, the polymeric matrix may comprise PCL in an amount of greater than 70% by weight, with respect to the total weight of the polymeric matrix, in particular greater than 80% by weight, and even more particularly greater than 90% by weight.

According to another embodiment, the polymeric matrix of the microparticles used in the pharmaceutical composition used according to the present invention comprises a mixture of at least a PCL and at least a PLGA.

In a particular embodiment, the pharmaceutical composition for use according to the present invention, is characterized by the microparticles, which are microparticles comprising colchicine and a polymeric matrix, wherein the polymeric matrix comprises at least a poly(lactic-co-glycolic acid) copolymer, at least poly(caprolactone) or at least a mixture of poly(lactic-co-glycolic acid) copolymer and poly(caprolactone).

In one embodiment, the polymeric matrix comprises poly(lactic-co-glycolic acid), copolymer poly(caprolactone) or a mixture of poly(lactic-co-glycolic acid) copolymer and poly(caprolactone) in an amount of greater than 70% by weight, with respect to the total weight of the polymeric matrix, in particular greater than 80% by weight, and even more particularly greater than 90% by weight.

According to said embodiment, the polymeric matrix may comprise a mixture of PCL and PLGA in an amount of greater than 70% by weight, with respect to the total weight of the polymeric matrix, in particular greater than 80% by weight, and even more particularly greater than 90% by weight.

The polymeric matrix may comprise one or more additional polymer(s), copolymer(s) or mixture thereof. Said additional polymer(s), copolymer(s) or mixture thereof may be present in the polymer matrix in an amount ranging from 0 to 30% by weight, in particular from 0 to 20% by weight, and more particularly from 0 to 10% by weight, with respect to the total weight of the polymeric matrix.

Non-limiting examples of suitable additional polymers or copolymers include poly(lactide) distinct from poly(lactic-co-glycolic acid) copolymer or PLA, poly(glycolide) distinct from poly(lactic-co-glycolic acid) copolymer or PGA, poly(lactide-co-caprolactone), poly(ethylene glycol), poly(ethylene oxide) or PEO, PLGA-b-PEO-b-PLGA, PLGA-b-PEO, polyhydroxyalkanoates, poly(hydroxybutyrate), poly(trimethylene carbonate), poly(dioxanone), poly(valerolactone), poly(alpha-hydroxy acids), poly(lactones), poly(amino acids), polyanhydrides, poly(orthoester), poly(acetals), polyurethanes, polythioesters, polyphosphoesters, poly(ester-co-amide), poly(vinyl alcohol) or PVA, PVA-g-PLGA, poly (ether ester) multiblock copolymers, polyvinyl pyrrolidone, poly(methacrylate), PEO-PPO-PEO (pluronics), gelatin, heparin, chondroitin sulfate; polysaccharides such as alginates, starch, chitosan and dextrans and any combinations thereof, and in particular selected from poly(lactide) distinct from poly(lactic-co-glycolic acid) copolymer and poly(caprolactone).

According to one embodiment, the drug loading content or percentage weight ratio between the colchicine and total weight of the microparticles ranges from 0.1% to 35%, in particular from 0.5 to 30%, even more particularly from 1 to 25%, for example from 2 to 20%.

In one embodiment, the controlled release microparticles according to the present invention are PLGA microspheres. In other words, in said embodiment, the polymeric matrix does not comprise any additional polymer or copolymer.

When PLGA copolymers are implemented as a polymer matrix, they can have a wide range of molecular weights and monomer ratios of lactic to glycolic acid, in particular of 75:25 to 50:50, and even more particularly of 50:50. Any suitable method known in the art of making the polymer can be used and the molecular weights may typically range from 5 to 150 kDa, in particular from 5 to 80 kDa, and more particularly from 5 to 50 kDa, for example from 10 to 150 kDa, from 10 to 80 kDa or from 10 to 50 kDa.

According to a further particular embodiment, the polymer forming the polymeric matrix comprises PLGA, for example in an amount of 100% by weight, with respect to the total weight of the polymeric matrix, and the PLGA is defined by:
A molecular weight between 5 and 80 kDa, and
A lactide:glycolide molar ratio of 75:25 to 50:50.

According to a more particular embodiment, the polymer forming the polymeric matrix comprises PLGA, for example in an amount of 100% by weight, with respect to the total weight of the polymeric matrix, and the PLGA is defined by:
A molecular weight between 5 and 50 kDa, and
A lactide:glycolide molar ratio of 50:50.

According to one embodiment, PLGA is either carboxylic or ester endcapped, and is in particular carboxylic endcapped.

According to another embodiment, microparticle matrix can comprise, and even may consist in, a blend of two PLGA copolymers, in particular one of low molecular weight and one of high molecular weight.

The microparticle may further comprise pharmaceutically acceptable excipients for modulating the drug release profile such as medium chain triglycerides, poly(oxyethylene) sorbitan fatty acid esters (e.g. polysorbate 20, polysorbate 80), sorbitan fatty acid esters, cyclodextrins, lecithin, mannitol, sucrose, inorganic salts and mixtures thereof.

The polymeric matrix of the microparticles may comprise one or more excipient(s). Said excipient(s) may be present in the polymer matrix in an amount not exceeding 15% by weight, in particular not exceeding 10% by weight, and more particularly not exceeding 5% by weight, with respect to the total weight of the polymeric matrix.

In one embodiment is provided a pharmaceutical composition comprising controlled release microparticles comprising colchicine, wherein the microparticles have a mean particle size greater than 10 μm, wherein said pharmaceutical composition presents an in vitro dissolution profile, wherein less than 25% of colchicine is released within 60 minutes, less than 50% of colchicine is released in 12 hours, wherein the colchicine is present in a concentration ranging from 2.5 to 2500 μg per ml of suspension and wherein the pharmaceutical composition has a volume ranging from 0.1 ml to 5 ml.

For the comfort of the patient and for safety reasons as explained above, it is advantageous to look for a pharmaceutical composition, suitable for intra articular injection into a joint, requiring only one injection able to relieve the patient thanks to this injection without needing multiple injections.

It is apparent from the illustrative part, and namely from example 8 that a controlled release is achieved fulfilling said requirements.

According to one embodiment, said pharmaceutical composition is further characterized in that it is effective to maintain systemic concentration of colchicine 12 hours after the intra-articular injection lower than 5 ng/ml, particularly lower than 2 ng/ml and more particularly lower than 1 ng/ml and to maintain synovial concentration of colchicine greater than 2 ng/ml, in particular comprised between 2 and 500 ng/ml, more particularly comprised between 2 and 250 ng/ml, more particularly comprised between 2 and 200 ng/ml, for example between 2 and 100 ng/ml, and even more particularly between 2 and 50 ng/ml.

The present invention further relates to a formulation under the form of a powder comprising controlled release microparticles comprising colchicine and a polymeric matrix, wherein the microparticles have a mean particle size equal or greater than 10 μm, and wherein the polymeric matrix comprises at least a poly(lactic-co-glycolic acid) copolymer, at least a poly(caprolactone) or at least a mixture of poly(lactic-co-glycolic acid) copolymer and poly(caprolactone), wherein the percentage weight ratio between the colchicine and the total weight of the microparticles ranges from 0.1 to 35% by weight, in particular from 0.5 to 30% by weight, more particularly between 1 and 25% by weight, and even more particularly between 2 and 20% by weight.

According to said aspect of the invention, the powder further comprises an immediate release dosage form containing colchicine, in particular wherein the maximal weight ratio of immediate release colchicine relative to the total amount of colchicine is 25%.

The present invention further relates to a pharmaceutical composition under the form of a sterile and injectable suspension suitable for an intra-articular injection, obtained by mixing a formulation under the form of a powder as described above, with an aqueous injection vehicle, wherein the pharmaceutical composition optionally comprises an excipient selected from a group consisting of a tonicity-enhancing agent, a wetting-agent, a viscosity-enhancing agent, a density-enhancing agent or a mixture thereof, and wherein said excipient may be present in the aqueous injection vehicle or in the powder.

In one embodiment, said pharmaceutical composition is characterized by the concentration in colchicine ranging from 0.05 to 5 mg per ml of suspension, in particular ranging from 0.1 to 4 mg per ml of suspension, in particular ranging from 0.1 to 2.5 mg per ml of suspension, more particularly from 0.25 to 2.5 mg per ml of suspension, and even more particularly from 0.5 to 1.5 mg per ml of suspension.

In one further embodiment, said pharmaceutical composition is characterized by the concentration in colchicine ranging from 5 to 2000 μg per ml of suspension, in particular from 5 to 1500 μg per ml of suspension, in particular from 5 to 1000 μg per ml of suspension, and more particularly from 5 to 500 μg per ml of suspension.

### Manufacturing Process for the Obtention of the Microparticles Comprising a Polymer Matrix

Any process suitable to produce polymeric microparticles with a mean particle size ranging from 10 μm to 100 μm is considered appropriate in the framework of the present invention.

Among such manufacturing process emulsification-based processes, such as high-pressure homogenization, for example using rotor-stator homogenizer in a batch or continuous mode, or membrane emulsification, followed by organic solvent removal by extraction/evaporation may be cited.

As far as the general principal of such methods is concerned, an emulsion may be prepared and processed over a membrane with pores with a determined size. The obtained microspheres can then be collected after extraction and evaporation of the organic solvent, washed and freeze-dried.

According to one particular embodiment, the microparticles may be manufactured from an O/W direct emulsion or from a W/O/W double emulsion technique.

According to one particular embodiment, the microparticles may be manufactured from a Solid-in-Oil-in-Water (S/O/W) double emulsion technique.

Schoubben, A., Ricci, M. & Giovagnoli, S. Meeting the unmet: from traditional to cutting-edge techniques for poly lactide and poly lactide-co-glycolide microparticle manufacturing. J. Pharm. Investig. 49, 381-404 (2019) reviews various methods to make PLGA microparticles that can be used in the framework of the present invention.

Piacentini, E. Pharmaceutical Particles Design by Membrane Emulsification: Preparation Methods and Applications in Drug Delivery. Curr. Pharm. Des. 23, 302-318 (2018) reviews various designs of such membrane emulsification technique, which may be used in the framework of the present invention.

Other manufacturing approaches suitable for obtaining the microparticles according to the present invention are atomization by spinning disk, atomization by spray-drying, a fluidized bed coating, or combinations thereof.

Alternatively, the microparticles may be manufactured using drop-on-demand, drop-by-drop and jet break-up processes such as inkjet printing or microfluidics.

Alternatively, the microparticles may be manufactured using supercritical fluid technologies.

Alternatively, the microparticles may be manufactured using microfabrication methods, such as template and mold-based techniques, such as soft lithography.

All these manufacturing processes are well known by the man skilled in the art.

The hereabove cited manufacturing processes are well known to the man skilled in the art.

### (ii) Multi Vesicular Liposomes

Multivesicular liposomes (MVL) are spherical particles with a mean diameter of 10-30 μm and composed of non-concentric multiple lipid bilayers arranged in a honeycomb-like structure. These lipid layers enclose numerous water-filled aqueous compartments that can be used to encapsulate a water-soluble drug such as colchicine.

MVLs are classically composed of at least one amphiphilic lipid and one neutral lipid. The amphiphilic lipid is selected from phospholipids for instance phosphatidylcholine or phosphatidylglycerol. The neutral lipid is selected from triglycerides having mono-unsaturated fatty acid ester moieties containing 14-18 carbons in the acyl chain (e.g. triolein, tripalmitolein), saturated fatty acid ester moieties containing 6 to 8 carbons in the acyl chain (e.g. tricaproin, tricaprylin) and mixtures thereof. Cholesterol can also be used in the composition.

MVLs are obtained by using a water-in-oil-in-water double emulsification process. In a first step, a water-in-oil emulsion is prepared by mixing phospholipids, triolein, tricaprylin and cholesterol solubilized in volatile and water-immiscible organic solvent with an aqueous solution containing the solubilized drug to be encapsulated. This first emulsion is then emulsified by mixing with a second aqueous solution to produce the water-in-oil-in-water emulsion. The energy needed to form the first and second emulsion can be provided either mechanically, by sonication or by combinations thereof. MVLs are finally obtained by removal of the volatile organic solvent from the double emulsion using gas stripping or flushing. Finally, removal of unencapsulated material, concentration of the MVLs and buffer exchange is performed using either diafiltration or cross-flow filtration system.

In one embodiment, the neutral lipids used to manufacture the MVL encapsulating colchicine comprises a mixture of triolein: tricaprylin with ratios ranging from 50:50 to 0:100.

### Pharmaceutical Composition

The pharmaceutical composition used in the framework of the present invention may take the form of a sterile and injectable composition, in particular a suspension composition, comprising an effective amount of colchicine.

By “sterile”, in the sense of the present invention, is meant an environment capable of guaranteeing to the considered compounds in a composition according to the invention safety requirements for the administration routes such as above-mentioned, notably into or through a joint. Indeed, for obvious reasons, it is essential that a composition according to the invention be devoid of any contaminant body capable of initiating an undesirable side reaction at the host site.

The pharmaceutical composition used in the framework of the present invention may be prepared with the formulation under the form of a powder comprising microparticles as described above. According to one embodiment, said pharmaceutical composition is a sterile and injectable composition for controlled release of colchicine, suitable for an intra-articular injection.

By its injectable character, a composition according to the invention necessarily comprises a physiologically acceptable medium, also called “aqueous injection vehicle”.

A “physiologically acceptable medium” means a medium devoid of toxicity and compatible with the injection and/or the application of the composition such as considered in the present invention.

The present invention more particularly relates to the pharmaceutical composition for use as defined above, wherein it is obtained by mixing a formulation under the form of a powder comprising controlled release microparticles comprising colchicine having a mean particle size equal or greater than 10 μm, with an aqueous injection vehicle, wherein the pharmaceutical composition optionally comprises an excipient selected from a group consisting of a tonicity-enhancing agent, a wetting-agent, a viscosity-enhancing agent, a density-enhancing agent or a mixture thereof, and wherein said excipient may be present in the aqueous injection vehicle or in the powder.

The composition may comprise a solvent or a mixture of physiologically acceptable solvents.

The composition may comprise a physiologically acceptable aqueous medium.

As an aqueous medium suitable for the invention, may be for example mentioned water.

As isotonic agents suitable for the preparation of a composition according to the invention, it may be mentioned sugars and sodium chloride.

The aqueous injection vehicle may in particular comprise a tonicity-enhancing agent, a wetting-agent, a viscosity-enhancing agent or a mixture thereof.

Among the tonicity-enhancing agent, the following may be cited: dextrose, mannitol, sorbitol, sucrose, glycerin, sodium chloride, potassium chloride, cyclodextrin and maltodextrin.

Among the wetting agents, the following may be cited: poly(oxyethylene) sorbitan fatty acid esters, such as commercially available under the trade name TWEEN, sorbitan fatty acid esters, such as commercially available under the trade name SPAN, poloxamer and lecithins.

Among the viscosity-enhancing agents, the following may be cited: sodium carboxymethyl cellulose (CMC), a glycosaminoglycan such as hyaluronic acid, dextran, collagen, poly(vinylpyrrolidone), poly(ethyleneglycol), gelatin, hydroxyethyl cellulose (HEC), methyl cellulose (MC), alginate, gum acacia, starch.

According to a particular embodiment, said pharmaceutical composition has a viscosity of from 5 to 1000 mPa·s, in particular from 5 to 500 mPa·s, in particular from 5 to 100 mPa·s and more particularly from 5 to 50 mPa·s at a shear rate of 10 s⁻¹.

According to one embodiment, said pharmaceutical composition may further comprise an immediate release dosage form containing colchicine. The presence of such immediate release dosage form aims at achieving a quick articular colchicine concentration so as to relieve pain quickly. In one embodiment, the maximal weight ratio of immediate release colchicine relative to the total amount of colchicine is 25%.

In certain embodiment of the invention, the weight ratio of immediate release colchicine relative to the total amount of colchicine may be between 0 and 5%, 0 and 10%, 0 and 15% or 0 and 20%.

In some embodiments, the length of release of the immediate release form is between 0 and 6 hours. In some embodiment, the length of release of the immediate release form is between 0 and 2 hours.

The immediate release dosage form may take the form of immediate fraction that are mixed with the controlled release microparticles as described above or be present in a continuous phase to which the microparticles are mixed when preparing the final formulation under the form of a powder, according to the present invention. Said continuous phase may of course comprise any further suitable pharmaceutically acceptable excipient than the ones already present within the microparticles.

### Additional Active Ingredients

Depending upon the particular condition, or disease, to be treated, additional therapeutic agents, which are normally administered to treat that condition, may be administered in combination with colchicine.

As used herein, the term “combination”, “combined,” and related terms refers to the simultaneous or sequential administration of said additional active ingredient with colchicine. For example, a combination may be administered with an additional active ingredient simultaneously or sequentially in separate unit dosage forms or together in a single unit dosage form.

According to one embodiment, hyaluronic acid may be injected into the painful joint, and for example be present in the powder or pharmaceutical composition according to the present invention. The use of hyaluronic acid in the treatment of arthritis, such as osteoarthritis, is well known, as viscosupplementation. Therefore, according to one embodiment, the present invention provides a pharmaceutical composition comprising colchicine according to the present invention further comprising hyaluronic acid.

In some embodiments, the present invention provides a pharmaceutical composition comprising colchicine according to the present invention further comprising at least an additional therapeutic agent. Suitable additional active ingredients are described in further detail below.

According to one embodiment, the following active ingredients may be combined, in particular any anti-inflammatory active ingredient:
(i) corticosteroid, such as prednisone, prednisolone, methylprednisolone, betamethasone, dexamethasone, triamcinolone acetonide, triamcinolone hexacetonide, budenoside, mometasone, ciclesonide, fluticasone, hydrocortisone, and pharmaceutically acceptable salts thereof, especially at low dose,
(ii) an NSAID, such as aspirin, diclofenac, aceclofenac, sulindac, ketorolac, ibuprofen, ketoprofen, naproxen, oxaprozine, flubiprofen, indomethacin, proglumetacin, tiaprofenic acid, meloxicam, piroxicam, tenoxicam, etodolac and celecoxib, etoricoxib, parecoxib, rofecoxib, valdecoxib, and pharmaceutically acceptable salts thereof,
(iii) an antiIL-1β agents such as anakinra, canakinumab, rilonacept, and pharmaceutically acceptable salts thereof,
(iv) Anti-IL-6 agents such as tocilizumab, siltuximab, sarilumab, and pharmaceutically acceptable salts thereof,
(v) an anti-TNFα agent, such as adalimumab, etanercept, infliximab, certolizumab, golimumab, and pharmaceutically acceptable salts thereof,
(vi) an anti-angiogenic agents such as bevacizumab, and pharmaceutically acceptable salts thereof,
(vii) an anti NGF (Nerve Growth Factors) agent, such as fasinumab, tanezumab, fulranumab, ABT-110, and pharmaceutically acceptable salts thereof,
(viii) an opioid, such as fentanyl, morphine, buprenorphine, hydromorphone, hydrocodone, oxycodone, meperidine, and pharmaceutically acceptable salts thereof,
(ix) an inhibitor of class I glucose transporters (e.g. GLUT-1/GLUT-3), such as cytochalasin B, WZB-117, STF-31, BAY-876
(x) mixtures thereof.

Said additional active ingredient may be formulated under immediate and/or controlled release dosage forms.

### Administration of the Composition

A pharmaceutical composition used in the framework of the present invention can be injected using any of the known methods in the art.

Particularly, said pharmaceutical composition may be administered by means of an injection device suitable for intra-articular injection such as a syringe equipped with a needle 19-29G, preferentially 22-29G, more preferentially 25-29G.

Throughout the description, including the claims, the expression “comprising a” should be understood as being synonymous with “comprising at least one” unless specifically stated otherwise.

The expressions “between . . . and . . . ” and “ranging from . . . to . . . ” should be understood to mean that the limits are inclusive, unless specified otherwise.

The following examples and figures are presented by way of non-limiting illustration of the invention.

### EXAMPLES

### Analytical Methods

Particle size was determined using optical microscopy (N=100 particles analyzed, Olympus BX51 with Stream Software).
Microparticle drug load was determined by dissolving 30 mg of particles with 2.5 mL acetonitrile in a 50 mL volumetric flask. After dissolution, 22.5 mL of methanol is added and mixed under vortexing. The flask was then filled up with water and thoroughly mixed using vortex. The medium was then centrifuged for 30 min at 4000 rpm and the supernatant is filtrated through a 0.45 μm PTFE filter (Acrodisc wwPTFE), discarding the first 2 mL. An aliquot of this solution is then analyzed by HPLC to determine the microparticle drug load.
Analysis by HPLC was conducted using a silica-based reversed-phase C8 column [GL Sciences, Inertsil C8-3, 4.6×250 mm] and a mobile phase [55% methanol, 45% of a 6.8 g/L KH₂PO₄ solution, solution adjusted to pH 5.5 with diluted phosphoric acid] at 1 mL/min flow rate with UV detection at 254 nm.
For the in vitro release tests from microparticles, aliquots of 10-30 mg microparticles were suspended in 50 mL of phosphate buffer (10 mM or 50 mM, pH 7.4) containing 0.02% sodium azide maintained at 37° C. in a horizontal shaking (incubator model 3033 commercialized by GFL). At different intervals, 3 mL of the media was removed and replaced with an equivalent amount of fresh media, and analyzed by UV at 350 nm.
Assay of colchicine in biological media (plasma and intraarticular lavages) was performed by LC-MS using a linear Ion Trap Quadrupole LC/MS/MS spectrometer equipped with a Kinetex C18 (100 mm×2.1 mm×1.7 μm) LC column and buffers [Buffer A: H₂O+FA 0.1% (Compressibility 0.45) and Buffer B: MeOH+FA 0.1% (Compressibility 1.25)] at a flow rate of 0.5 ml/min.
For assay of colchicine in plasma, sampled blood was centrifuged at 1300G for 12 minutes at 4° C. Aliquots of 50 μl of recovered plasma were placed in microtiter plate and 100 μl of MeOH added in each well. centrifugation was performed at 2200G for 12 minutes and the supernatant recovered. SPE processing was performed prior to running the LC-MS analysis.
For assay of free colchicine in intra-articular lavages, the microparticles were removed from the biological samples to ensure assay of the released colchicine solely. This was done by filtration through vacuum. The recovered samples were then treated as was done for the plasma samples.

### Example 1: Sustained-Release Colchicine Microparticles

An organic solvent phase was prepared by solubilizing 0.23 g of colchicine (Indena, Italy) and 0.91 g of PLGA Resomer RG 503H (Evonik Industries AG, Essen, Germany) in 2.51 g of dichloromethane (Merck) under vortexing.

Emulsification step was performed by adding 5 g of a PVA solution at 1% (Mowiol 4-88, Sigma-Aldrich) to the organic phase under high shear emulsification (Heidolph homogenizer DIAX 900, 11600 rpm) during 1 minute.

Emulsion was then poured into a hardening bath (0.5 L of an aqueous PVA solution at 1%) and stirred for 3 hours at 300 rpm with an overhead stirrer.

The solid particles were then separated from aqueous phase by pouring the emulsion over a stainless-steel screen with 12 μm pore diameter and washed three times with 50 mL of distilled water.

The microparticles were then transferred to glass dishes, dried at 5° C. under 7 mbar pressure for 18 hours and stored in amber glass vials.

Analytical tests, including the in vitro profile of said microparticles were then preformed. The results are gathered in table 3 below.

TABLE 3
Analytical results for Colchicine PLGA microparticles

| | | In vitro release in |
|---|---|---|
| Drug loading content | Particle size | 50 mM PBS, pH 7.4 |
| (colchicine wt %) | (Dv, μm) | (%) |
| 12 | D10 = 20 μm | 1 day: 8.4 |
| | D90 = 87 μm | 2 days: 15 |
| | Mean Particle | 3 days: 24.3 |
| | Size = 35 μm | 7 days: 73 |
| | | 10 days: 89.8 |
| | | 13 days: 90.4 |

In this example, colchicine-loaded PLGA microparticles were prepared with a mean particle size of 35 μm and releasing 80% of its drug content after 7 days.

### Example 2: Sustained-Release Colchicine Microparticles

An organic solvent phase was prepared by solubilizing 0.7 g of colchicine (Indena, Italy) and 1.05 g of sterile PLGA Resomer RG 503H (Evonik Industries AG, Essen, Germany) in 2.5 g of dichloromethane (Merck) under vortexing.

Emulsification step was performed in a laminar flow hood by adding 5 g of a sterile PVA solution at 1% (Mowiol 4-88, Sigma-Aldrich) to the organic phase under high shear emulsification (Heidolph homogenizer DIAX 900, 11600 rpm) during 1 minute.

Emulsion was then poured into a hardening bath (0.5 L of an aqueous PVA solution at 1% prepared using sterile water for injection) and stirred for 3 hours at 300 rpm with an overhead stirrer.

The solid particles were then separated from aqueous phase under laminar flow by pouring the emulsion over a stainless-steel screen with 12 μm pore diameter and washed three times with 50 mL of sterile water for injection.

The microparticles were then transferred to glass dishes, dried at 5° C. under 7 mbar pressure for 18 hours and stored in sealed amber glass vials at 5° C.

Analytical tests, including the in vitro profile, were performed as in Example 1 and are shown in Table 4.

TABLE 4
Analytical results for Colchicine PLGA microparticles

| | | In vitro release in |
|---|---|---|
| Drug loading content | Particle size | 50 mM PBS, pH 7.4 |
| (colchicine wt %) | (Dv, μm) | (%) |
| 26 | D10 = 11 μm | 0.13 day: 19.7 |
| | D90 = 25 μm | 1 day: 45.8 |
| | Mean particle | 2 days: 56.1 |
| | size = 17 μm | 5 days: 85.7 |
| | | 8 days: 94.4 |

In this example, colchicine-loaded PLGA microparticles were prepared with a mean particle size of 17 μm and releasing 80% of its drug content between 2 days and 5 days.

### Example 3: Sustained-Release Colchicine Microparticles

An organic solvent phase was prepared by solubilizing 0.7 g of colchicine (Indena, Italy), 0.79 g of PLGA Resomer RG 503H and 0.26 g of PLGA Resomer RG 502H (Evonik Industries AG, Germany) in 2.52 g of dichloromethane (Merck) under vortexing.

Emulsification step was performed by adding 5 g of a PVA solution at 1% (Mowiol 4-88, Sigma-Aldrich) to the organic phase under high shear emulsification (Heidolph homogenizer DIAX 900, 11600 rpm) during 1 minute.

Emulsion was then poured into a hardening bath (0.5 L of an aqueous PVA solution at 1%) and stirred for 3 hours at 300 rpm with an overhead stirrer.

The solid particles were then separated from aqueous phase by pouring the emulsion over a stainless-steel screen with 12 μm pore diameter and washed three times with 50 mL of distilled water.

The microparticles were then transferred to glass dishes, dried at 5° C. under 7 mbar pressure for 18 hours and stored in amber glass vials.

Analytical tests, including the in vitro profile, were performed as in Example 1 and are shown in Table 5.

TABLE 5
Analytical results for Colchicine PLGA microparticles

| | | In vitro release in |
|---|---|---|
| Drug loading content | Particle size | 50 mM PBS, pH 7.4 |
| (colchicine wt %) | (Dv, μm) | (%) |
| 19 | D10 = 14 μm | 0.13 day: 46 |
| | D90 = 44 μm | 1 day: 79.1 |
| | Mean particle | 2 days: 87 |
| | size = 32 μm | |

In this example, colchicine-loaded PLGA microparticles were prepared with mean particle size of 32 μm and releasing 80% of its drug content between 1 day and 2 days.

### Example 4: Sustained-Release Colchicine Microparticles—Continuous Emulsification Using a High-Shear In-Line Mixer

Microparticles were prepared using Silverson L5M-A high shear in-line mixing assembly (SILVERSON Machines, East Longmeadow, Massachusetts, U.S.A.) equipped with an emulsion stator screen with fine 0.8 mm perforations. Bottom inlet port of the mixing chamber was equipped with a male run tee connected to two different perilstatic pumps through a 1 mm diameter Viton tubing and a 5 mm diameter Viton tubing. Outlet port was equipped with a 5 mm diameter Viton tubing.

The dispersed phase was prepared by dissolving 25 g of PLGA copolymer (Resomer RG503H, Evonik Industries AG, Germany) and 4.4 g of colchicine in 100 g of ethyl acetate (Alfa Aesar).

Separately, a continuous phase was prepared by adding 74.9 g of ethyl acetate to 998 g of an aqueous solution containing lwt % PVA.

For the emulsification step, the Silverson speed was set at 2000 rpm and the dispersed phase was fed through the 1 mm diameter inlet tube at a flow rate of about 20 g/min and the continuous phase was fed simultaneously through the 5 mm diameter inlet tube at a flow rate of about 200 g/min.

Resulting emulsion flowing from the outlet port was added to a beaker containing a 6 L water solution used to extract the organic solvent and that was being stirred at 300 rpm with a laboratory mixer.

After 2 hours of extraction duration, the resulting suspension was passed through a stainless-steel screen with 12 μm pore diameter, rinsed three times with 150 mL of distilled water and finally dried by lyophilization.

### Example 5: Pharmaceutical Composition—Preparation of Injectable Suspension

An aqueous injection vehicle was prepared using pyrogen-free excipients and consisting of 1.4% of low viscosity sodium carboxymethyl cellulose (Aqualon CMC 7LF PH BET, Ashland), 0.1% Polysorbate 20 (Acros Organics), 0.13% disodium hydrogen phosphate dihydrate, 0.1% citric acid (Roth) and 0.65% sodium chloride (Roth). Final pH of the solution was adjusted to 7.2 using concentrated sodium hydroxide solution (Roth). The vehicle was then autoclaved at 121° C. for 15 minutes (MultiControl 2, CertoClav) and 5 mL aliquots of the solution transferred aseptically into 10 mL vials under a laminar air flow bench.

A 20 mg aliquot of microparticles as prepared according to example 2 was dispersed in each vial using a vortex mixer

This pharmaceutical composition is intended for intra-articular injection to treat crystal-associated and non-crystal associated acute inflammatory arthritis, in particular non-crystal associated acute arthritis, such as Rheumatoid arthritis, Ankylosing spondylitis, Juvenile arthritis, Psoriatic arthritis, Lupus or crystal-associated arthropathies attacks or flares, with an injection volume to be adapted to the particular intra-articular injection site.

In an example, a volume of 0.25 mL containing a 0.26 mg colchicine dose can be injected into the first metatarsophalangeal joint using a 27 G gauge needle.

In another example a volume of 2.5 mL containing a 2.59 mg colchicine dose can be injected into the knee joint using a 23 G gauge needle.

### Example 6: Sustained-Release Colchicine Microparticles and Pharmaceutical Composition Comprising it

Microparticles were prepared as follows: A 1 wt % polyvinyl alcohol (PVA) solution was prepared by heating 2970 g water to 70° C. 30 g of PVA was dispersed therein under magnetic agitation and the preparation allowed to cool down prior to further use.

4 g of colchicine was solubilized in 14.3 g methylene chloride using vortex agitation. 6 g of PLGA 50:50 Resomer 503H was then introduced in the mixture and vortexed until dissolved to form a polymer-drug solution.

28.6 g of the previously prepared PVA 1 wt % solution was added to the drug-polymer solution over 30 seconds under high shear (11 600 RPM) to form a pre-emulsion. A Heidolph Diax 900 rotor stator mixer equipped with a 10G head was used.

Agitation was continued for another 30 seconds after which, the pre-emulsion was poured at once into a hardening bath containing 2860 g of the PVA 1 wt % solution previously prepared.

Agitation was maintained using an IKA 10 cm 4 bladed propeller stirrer with an agitation speed set at 250 RPM for 3 hours until methylene chloride is evaporated.

The formed microparticles were centrifuged at 4000 RPM for 3 minutes using a swinging bucket centrifuge apparatus in order to remove the main portion of the water phase and get a concentrated particle slurry prior separation per cellulose filter (12.5 cm diameter).

The microparticles were then rinsed with 200 ml water.

The washed microparticles were stored in a glass dish over molecular sieve overnight and then dried under vacuum in a 2-4 LSCplus Epsilon set at 5 mBar and 5° C. shelf temperature for 18 hours.

The collected microparticles were then stored at 5° C.

Analytical tests, including the in vitro profile, were performed as in Example 1 and are shown in Table 6.

TABLE 6
Analytical results for Colchicine PLGA microparticles

| Colchicine | |
|---|---|
| loading content | Particle size |
| (wt %) | (Dv, μm) |
| 31.1 | D10 = 20 μm |
| | D90 = 35 μm |
| | Mean Particle |
| | size = 29 μm |

In this example, colchicine-loaded PLGA microparticles were prepared with mean particle size of 29 μm and a drug loading content of 31.1 wt %.

An aqueous injection vehicle was prepared under laminar flow using pyrogen-free excipients and consisting of 1.4% of low viscosity sodium carboxymethyl cellulose (Aqualon CMC 7LF PH BET, Ashland), 0.1% Polysorbate 20 (Acros Organics), 0.13% disodium hydrogen phosphate dihydrate, 0.1% citric acid (Roth) and 0.65% sodium chloride (Roth). Final pH of the solution was adjusted to 7.2 using concentrated sodium hydroxide solution (Roth). The vehicle was then autoclaved at 121° C. for 15 minutes (MultiControl 2, CertoClav) and 2 mL aliquots of the solution transferred aseptically into 5 mL vials under a laminar air flow bench.

15 min prior to in vivo injection as described in following example 7, 16 mg aliquot of microparticles as prepared according to this example was dispersed in each vial containing 2 mL of injection vehicle using a vortex mixer under laminar flow until homogeneous dispersion was obtained. The colchicine-loaded PLGA microparticles suspension thus prepared yielded a colchicine concentration of 2.49 mg/ml (or 2490 μg/ml).

### Example 7: In Vivo Release Behavior

This example shows the colchicine concentrations assayed in both rabbit articular lavage and plasma after injection of colchicine-loaded PLGA microparticles manufactured in Example 6 in an inflamed rabbit knee joint.

### Material and Method

Male New Zealand rabbits aged 75 days were used for this experiment. Knee joint inflammation was triggered by the injection of 25 mg Monosodium Urate Crystals dispersed in 400 μL Phosphate Buffered Saline. Right after crystals injection, a 200 μL volume of the colchicine-loaded PLGA microparticles suspension manufactured as described in example 6 was injected into the inflamed rabbit knee joint, thus conveying a colchicine dose of about 0.5 mg.

Blood was collected at T2h, T6h and daily from T24h to T120h to quantify free colchicine in plasma.

Articular lavage was collected from animals at T6h, T24h, T72h and T120h to quantify free colchicine released from the microparticles. Here, 4 rabbits were euthanized at each time point.

### Results

Assay on both types of biological samples was performed by LC-MS according to the method as described above and the results shown in the Tables 7 and 8 below.

TABLE 7
Colchicine assay in rabbit articular lavage (LC-MS)

| | Colchicine assay in | Number of |
|---|---|---|
| Time | articular lavage | animals tested |
| (Hours) | (ng/ml) Mean | per time point |
| 6 | 221.75 | 4 |
| 24 | 302.56 | 4 |
| 72 | 148.22 | 4 |
| 120 | 435.30 | 4 |

TABLE 8
Free Colchicine assay in plasma (LC-MS)

| | Colchicine assay | Number of |
|---|---|---|
| Time | in plasma | animals tested |
| (Hours) | (ng/ml) Mean | per time point |
| 2 | 1.023 | 16 |
| 6 | 0.552 | 16 |
| 24 | 0.297 | 12 |
| 48 | 0.154 | 8 |
| 72 | 0.198 | 8 |
| 96 | 0.474 | 4 |
| 120 | 0.699 | 4 |

### CONCLUSION

This example shows that a pharmaceutical composition under the form of a suspension according to the invention is able to maintain a systemic concentration of colchicine after administration of sustained release colchicine microparticles by intra-articular injection below 1.5 ng/ml whereas a synovial concentration of more than 100 ng/ml may be maintained. These results may be extrapolated to select a human dosage achieving similar concentrations ratios and thresholds in humans. This extrapolation may be done using, allometric scaling where the dose is based on normalization by body surface area using classical approach such ones recommended by Health Authorities, or by targeting similar local concentration depending on targeted joint.

Without to be bound by any theory and based on a dose of 0.5 mg administered in rabbits (average animal weight of 1.8 kg), the human equivalent dose would range from 6.3 mg or 6300 μg (reference body weight of 70 kg as calculated based on body surface area allometric scaling as exemplified in Nair, A.; Jacob, S. (2016). A simple practice guide for dose conversion between animals and human, Journal of Basic and Clinical Pharmacy), 2.7 mg or 2700 μg (targeting similar local concentration in human knee joint) and 0.9 mg or 900 μg (targeting similar local concentration in human knee joint combined with allometric scaling). These calculated doses in human are in line with the medium/large joints doses as reported in Table 1 above.

Finally said example demonstrates that in human, a pharmaceutical composition under the form of a suspension according to the invention is able to maintain a systemic concentration of colchicine after administration of sustained release colchicine microparticles by intra-articular injection below 5 ng/ml particularly lower than 2 ng/ml and more particularly lower than 1 ng/ml and to maintain synovial concentration of colchicine greater than 2 ng/ml, in particular comprised between 2 and 500 ng/ml whereas a synovial concentration of more than 6 ng/ml may be maintained.

### Example 8: Stability and In Vitro Release Profile

### Material and Method

Stability of dry colchicine microparticles described in example 6 was tested after 5 months of storage at 5° C.

The in vitro release profile was then assessed as in examples 1, 2 and 3 above. The in vitro release profiles were performed two days after manufacturing, then after 5 months of storage at 5° C.

### Results

The sustained release microparticles of colchicine were stated as being stable.

The release data are represented in Table 9 below.

TABLE 9
release data before and after storage

| | Colchicine in vitro release | Colchicine in vitro release |
|---|---|---|
| | in 50 mM PBS, pH 7.4 | in 50 mM PBS, pH 7.4 |
| | - (%=)- | - (%) - |
| | Dissolution performed | Dissolution performed |
| Time | two days after | after 5 months storage |
| (Days) | manufacturing (n = 2) | at 5° C. (n = 2) |
| 0.13 | 1.7 | 2.9 |
| 1 | 16.5 | 16.6 |
| 2 | 24.7 | 24.7 |
| 4 | 43.6 | 43.1 |
| 7 | 86.2 | 69.0 |
| 11 | 93.2 | 82.6 |

### CONCLUSION

It may be concluded that the microparticles exhibit comparable dissolution profiles after 5 months of storage at 5° C. and are conform to the requirements of the invention in terms of release properties.

### Example 9: Sustained-Release Colchicine Microparticles and Pharmaceutical Composition Comprising it

Microparticles were prepared as follows: A 1 wt % polyvinyl alcohol (PVA) solution was prepared by heating 2970 g water to 70° C. 30 g of PVA was dispersed therein under magnetic agitation and the preparation allowed to cool down prior to further use.

4.03 g of colchicine was solubilized in 10.75 mL methylene chloride using vortex agitation. 6.02 g of PLGA 50:50 Resomer 503H was then introduced in the mixture and vortexed until dissolved to form a polymer-drug solution.

28.6 g of the previously prepared PVA 1 wt % solution was added to the drug-polymer solution over 60 seconds under high shear (11 000 RPM) to form a pre-emulsion. An IKA T25 Ultra-Turrax rotor stator mixer equipped with a S25N 10G head was used.

The pre-emulsion was poured slowly into a hardening bath containing 2860 g of the PVA 1 wt % solution previously prepared.

Agitation was maintained using a double propeller stirrer with an agitation speed set at 300 RPM for 3 hours until methylene chloride is evaporated.

The formed microparticles were centrifuged at 4000 RPM for 3 minutes using a swinging bucket GT2R centrifuge apparatus in order to remove the main portion of the water phase and get a concentrated particle slurry prior separation per 12-25 μm Whatman cellulose filter.

The microparticles were then rinsed with 150 ml water.

The washed microparticles were then vacuum dried at 0.08 mBar and 15° C. shelf temperature for 17 hours.

The collected microparticles were then stored at 5° C.

Analytical tests, including the in vitro profile, were performed as in Example 1 and are shown in Table 10.

TABLE 10
Analytical results for Colchicine PLGA microparticles

| | | In vitro release |
|---|---|---|
| Colchicine | | in 10 mM PBS, |
| loading content | Particle size | pH 7.4 |
| (wt %) | (Dv, μm) | n = 3 (%) |
| 22 | D10 = 6 μm | 0.08 day: 7.9 |
| | D90 = 37 μm | 1 day: 29.1 |
| | Mean particle | 2 days: 39.9 |
| | size = 15 μm | 3 days: 48.8 |
| | | 4 days: 55.8 |
| | | 7 days: 72.2 |

In this example, colchicine-loaded PLGA microparticles were prepared with mean particle size of 15 μm and a drug loading content of 22.02 wt %.

An aqueous injection vehicle was prepared under laminar flow using pyrogen-free excipients and consisting of 1.4% of low viscosity sodium carboxymethyl cellulose (Aqualon CMC 7LF PH BET, Ashland), 0.1% Polysorbate 20 (Emprove Essential from Merck), 0.13% disodium hydrogen phosphate dihydrate (Roth), 0.1% citric acid (Roth) and 0.65% sodium chloride (Roth). Final pH of the solution was adjusted to 6.9 using concentrated sodium hydroxide solution (Roth). The vehicle was then autoclaved at 121° C. for 20 minutes (Systec 3150EL) and 55 mL aliquots of the solution transferred aseptically into 100 mL vials under laminar air flow.

15 min prior to in vivo injection as described in following example 10, a 10 mg aliquot of microparticles as prepared according to this example was dispersed in 55 mL of injection vehicle using sonication for 30 seconds until homogeneous dispersion was obtained. The colchicine-loaded PLGA microparticles suspension thus prepared, yielded a colchicine concentration of 0.04 mg/ml or 40 μg/ml.

### Example 10: Efficacy on Pain—In Vivo Studies

This example shows the effect of colchicine-loaded PLGA microparticles on pain in an inflamed ankle joint in a rat model.

### Material and Method

4 weeks old male Sprague Dawley rats were used for this experiment. The effect of a 2 μg dose of encapsulated colchicine in an inflamed joint ankle was evaluated.

The rats were randomly separated into 2 groups of 9 animals each, as detailed in Table 11 below:

TABLE 11
Groups characteristics for pain evaluation

| | | Injection 1 | | | Injection 2 |
|---|---|---|---|---|---|
| | | | | Injected | Inflammation |
| | Group | | Injection | colchicine | of the ankle |
| | name | Treatment | Volume | dose | joint |
| 1 | PBS-Carra | PBS | 50 μl | Not | Carrageenan |
| | | | | applicable | 1 mg |
| 2 | Colch- | Colchicine- | 50 μl | 2 μg | Carrageenan |
| | Carra | loaded | | | 1 mg |
| | | PLGA | | | |
| | | microparticles | | | |
| | | suspension | | | |
| | | prepared in | | | |
| | | Example 9 | | | |

Carrageenan and PBS were supplied by Sigma Aldrich.

In each group, the animals were first injected with the treatment as described in Table 11 above, then the inflammatory material was injected. A delay of less than 5 minutes was measured between the two injections.

Ankle joint inflammation was triggered by the injection of 1 mg Carrageenan dispersed in 30 μL Phosphate Buffered Saline. All the injections were made in the left posterior ankle of the animals.

Pain evaluation was performed using Von Frey hairs (Bioseb, France). These were presented perpendicular to the plantar surface with sufficient force to cause slight buckling against the paw. Stimuli were presented at intervals of several seconds, allowing for apparent resolution of any behavioural responses to previous stimuli. A positive response was noted if the paw was sharply withdrawn. Flinching immediately upon removal of the hair was also considered a positive response. Ambulation was considered an ambiguous response, and in such cases the stimulus was repeated. Von Frey hairs were presented in ascending order of strength (0.4; 0.6; 1; 1.4; 2; 4; 6; 8; 10; 15; 26). Each hair was presented 5 times and held each time for 5 seconds. For each rat, ascending stimuli were tested either until a hair strength was reached that caused 70% response.

Basal reaction of the animals was evaluated 3 days prior to initiating the experiments (D-3). Both left and right posterior plantar surfaces were evaluated.

After treatment administration (DO), pain in both left (inflamed/treated) and right (control) plantar surfaces were evaluated 2 hours (D0+2h), 6 hours (D0+6h) and 24 hours (D1) after the injections. The collected data were normalized with respect to the basal reaction at D-3.

### Results

The results presented in FIG. 1, show that a 2 μg colchicine dose conveyed via PLGA microparticles injected directly into the rat ankle joint, significantly reduced the pain induced by carrageenan-triggered inflammation of said joint. Pain decrease is observed at 2 hours and remains mainly suppressed at 6 and 24 hours.

To assess if the difference between normalized groups data is statistically significant, the normality and homoscedasticity of each group and at each timepoint has been tested first on raw and log-transformed data. Then, a one-way analysis of variance (ANOVA) or a Kruskal-Wallis Rank Sum test has been performed followed by Dunnett' multiple comparison test to take into account for multiplicity.

At 2 hours (D0+2h), 6 hours (D0+6h) and 24 hours (D1) after injection, the Von Frey hair threshold difference between the group injected at 2 μg Colchicine (Colch-Carra group) and the PBS control group (PBS-Carra group) are statistically significant (“D0+2h” p-value <0.05, “D0+6h” p-value <0.01, “D1” p-value <0.05). These statistically significant results are obtained similarly by using other multiple testing procedures such as Tukey Honest Significant Differences. Results are also confirmed with Log-transformed data to take into account non-linearity of Von Frey measurements.

Without to be bound by any theory and based on a dose of 2 μg administered in rats (average animal weight of 250 g), the human equivalent dose would range from 0.33 mg or 330 μg (reference body weight of 70 kg as calculated based on body surface area allometric scaling as exemplified in “Nair, A.; Jacob, S. (2016). A simple practice guide for dose conversion between animals and human, Journal of Basic and Clinical Pharmacy”, or targeting similar local concentration in human knee joint) to 15 μg (targeting similar local concentration in human knee joint combined with allometric scaling). These calculated doses in human are in line with the medium/large joints doses as reported in Table 1 above.

1-55. (canceled)

56. A method of treating a crystal-associated or non-crystal associated acute inflammatory arthritis in a patient in need thereof, comprising at least administering, to a painful joint of the patient, a pharmaceutical composition in the form of a suspension comprising controlled release microparticles comprising colchicine, wherein:
the step of administering is performed by intra-articular injection of said pharmaceutical composition into the painful joint,
the controlled release microparticles have a mean particle size greater than 10 μm,
the time required to release 80% by weight of the colchicine is greater than 1 day and up to 15 days,
the colchicine is present in a concentration ranging from 2.5 to 2500 μg per ml of suspension, and
the pharmaceutical composition has a volume ranging from 0.1 ml to 5 ml.

57. The method according to claim 56, wherein the colchicine is present in a concentration ranging from 5 to 2000 μg per ml of suspension.

58. The method according to claim 56, wherein the colchicine is present in a concentration ranging from 5 to 500 μg per ml of suspension.

59. The method according to claim 56, wherein the microparticles are microparticles comprising a polymeric matrix or multivesicular liposomes.

60. The method according to claim 56, wherein the microparticles are microparticles comprising colchicine and a polymeric matrix, and wherein the polymeric matrix comprises at least a poly(lactic-co-glycolic acid) copolymer, at least poly(caprolactone) or at least a mixture of poly(lactic-co-glycolic acid) copolymer and poly(caprolactone).

61. The method according to claim 56, wherein the crystal-associated and non-crystal associated acute inflammatory arthritis is selected from non-crystal associated acute arthritis and crystal-associated arthropathy attacks or flares or acute forms of tendinitis and capsulitis.

62. The method according to claim 56, wherein the crystal-associated acute inflammatory arthritis is selected from rheumatoid arthritis, ankylosing spondylitis, juvenile arthritis, psoriatic arthritis and lupus.

63. The method according to claim 56, wherein the non-crystal associated acute inflammatory arthritis is selected from gout, chondrocalcinosis, calcifying tendinitis and Milwaukee shoulder syndrome.

64. The method according to claim 56, wherein the painful joint is selected from a metatarsophalangeal joint, a metacarpophalangeal joint, a knee joint, a shoulder joint, a wrist joint, an elbow joint, an ankle joint, a hip joint, a vertebral joint and a midfoot joint.

65. The method according to claim 56, wherein the time required to release 80% by weight of the colchicine is more than 1 day and up to 10 days.

66. The method according to claim 56, wherein the time required to release 80% by weight of the colchicine is from 2 days to 15 days.

67. The method according to claim 56, wherein the time required to release 80% by weight of the colchicine is from 2 days to 7 days.

68. The method according to claim 56, wherein less than 50% by weight of colchicine is released at 12 hours after the administration.

69. The method according to claim 56, wherein the pharmaceutical composition is effective to maintain a systemic concentration of colchicine lower than 5 ng/ml and a synovial concentration of colchicine greater than 2 ng/ml for 12 hours after the intra-articular injection.

70. The method according to claim 56, wherein the pharmaceutical composition is effective to maintain a systemic concentration of colchicine lower than 2 ng/ml and a synovial concentration of colchicine between 2 and 50 ng/ml 12 hours after the intra-articular injection.

71. The method according to claim 56, wherein the microparticles have a mean particle size lower than 100 μm.

72. The method according to claim 56, wherein the microparticles have a mean particle size of between 10 and 40 μm.

73. The method according to claim 56, wherein the pharmaceutical composition is obtained by mixing a powder comprising the controlled release microparticles comprising colchicine having a mean particle size equal or greater than 10 μm, with an aqueous injection vehicle, wherein the pharmaceutical composition optionally comprises an excipient selected from the group consisting of a tonicity-enhancing agent, a wetting-agent, a viscosity-enhancing agent, and a density-enhancing agent or a mixture thereof, and wherein said excipient is present in the aqueous injection vehicle or in the powder.

74. The method according to claim 56, wherein the pharmaceutical composition has a viscosity of from 5 to 1000 mPa·s at a shear rate of 10 s⁻¹.

75. The method according to claim 56, wherein said pharmaceutical composition further comprises an immediate release dosage form containing colchicine.

76. The method according to claim 60, wherein the polymeric matrix further comprises one or more additional polymer(s) or copolymer(s) selected from a poly(lactide) that is not a poly(lactic-co-glycolic acid) copolymer, a poly(glycolide) that is not poly(lactic-co-glycolic acid) copolymer, a poly(lactide-co-caprolactone), a poly(ethylene glycol), poly(ethylene oxide), a PLGA-b-PEO-b-PLGA, PLGA-b-PEO, a polyhydroxyalkanoate, a poly(hydroxybutyrate), a poly(trimethylene carbonate), a poly(dioxanone), a poly(valerolactone), a poly(alpha-hydroxy acid), a poly(lactone), a poly(amino acid), a polyanhydride, a poly(orthoester), a poly(acetal), a polyurethane, a polythioester, a polyphosphoester, a poly(ester-co-amide), a poly(vinyl alcohol), a PVA-g-PLGA, a poly (ether ester), a multiblock copolymer, a polyvinyl pyrrolidone, a poly(methacrylate), a PEO-PPO-PEO, gelatin, heparin, chondroitin sulfate, a polysaccharide and/or combinations thereof.

77. The method according to claim 60, wherein the polymeric matrix comprises poly(lactic-co-glycolic acid), copolymer poly(caprolactone) or a mixture of poly(lactic-co-glycolic acid) copolymer and poly(caprolactone) in an amount of greater than 70% by weight, with respect to the total weight of the polymeric matrix.

78. The method according to claim 56, wherein a drug loading content or a percentage weight ratio between the colchicine and a total weight of the microparticles ranges from 0.1% to 35%.

79. The method according to claim 78, wherein the drug loading content or the percentage weight ratio between the colchicine and the total weight of the microparticles ranges from 2 to 20%.

80. The method according to claim 56, wherein a volume of the pharmaceutical composition is adapted to the joint, and ranges from 2 to 5 ml for a shoulder, from 1 to 2 ml for a vertebral joint, from 0.1 to 1 ml for a midfoot joint, from 0.1 to 0.5 ml for a metatarsophalangeal joint, from 0.1 to 0.5 ml for a metacarpophalangeal joint, from 2 to 5 ml for a knee joint, from 0.5 to 1 ml for a wrist joint, from 2 to 5 ml for an elbow joint, from 1 to 3 ml for an ankle joint and from 2 to 5 ml for a hip.

81. The method according to claim 56, wherein the patient has renal and/or hepatic impairment and is simultaneously, separately or sequentially treated by at least one active ingredient selected from: atazanavir, clarithromycin, darunavir, ritonavir, indinavir, itraconazole, ketoconazole, lopinavir, ritonavir, nefazodone, nelfinavir, ritonavir, saquinavir, telithromycin, tipranavir, amprenavir, aprepitant, diltiazem, erythromycin, fluconazole, fosamprenavir, verapamil, cyclosporine and ranolazine.

82. A pharmaceutical composition comprising controlled release microparticles comprising colchicine, wherein:
the microparticles have a mean particle size greater than 10 μm,
said pharmaceutical composition presents an in vitro dissolution profile in which less than 25% of the colchicine is released within 60 minutes and less than 50% of the colchicine is released in 12 hours,
the colchicine is present in a concentration ranging from 2.5 to 2500 μg per ml of suspension, and
the pharmaceutical composition has a volume ranging from 0.1 ml to 5 ml.

83. A powder comprising controlled release microparticles comprising colchicine and a polymeric matrix,
wherein:
the controlled release microparticles have a mean particle size equal to or greater than 10 μm,
the polymeric matrix comprises at least a poly(lactic-co-glycolic acid) copolymer, a poly(caprolactone) or a mixture of poly(lactic-co-glycolic acid) copolymer and poly(caprolactone),
and a percentage weight ratio between the colchicine and a total weight of the microparticles ranges 0.1 to 35% by weight.

84. The powder according to claim 83, wherein the powder further comprises an immediate release dosage form containing colchicine.

85. A pharmaceutical composition in the form of a sterile and injectable suspension suitable for an intra-articular injection, wherein:
the pharmaceutical composition is obtained by mixing the powder according to claim 83 with an aqueous injection vehicle,
the pharmaceutical composition optionally comprises an excipient selected from the group consisting of a tonicity-enhancing agent, a wetting-agent, a viscosity-enhancing agent, a density-enhancing agent or a mixture thereof, and
said excipient is present in the aqueous injection vehicle or in the powder.

86. The pharmaceutical composition according to claim 85, wherein the colchicine is present in a concentration ranging from 5 to 2000 μg per ml of the sterile and injectable suspension.

87. The pharmaceutical composition according to claim 85, wherein the colchicine is present in a concentration ranging from 5 to 500 μg per ml of the sterile and injectable suspension.
